(19) United States
(12) Patent Application Publication
(10) Pub. No.: US 2024/0050727 A1
(43) Pub. Date: Feb. 15, 2024

(54) DEVICES FOR FORMING IN SITU MICRONEEDLES AND METHODS THEREOF

(71) Applicant: University of Connecticut, Farmington, CT (US)

(72) Inventors: Ali Tamayol, Farmington, CT (US); Mohamadmahdi Samandari, Farmington, CT (US); Steven Toro, Farmington, CT (US); Jacob Quint, Farmington, CT (US)

(21) Appl. No.: 18/449,499

(22) Filed: Aug. 14, 2023

Related U.S. Application Data
(60) Provisional application No. 63/397,690, filed on Aug. 12, 2022.

(51) Int. Cl. A61M 37/00

(52) U.S. Cl. CPC A61M 37/0015; A61M 2205/3368; A61M 2205/106; A61M 2205/3633; A61M 2037/003; A61M 2207/10

(57) ABSTRACT

A device and method for generating in-situ microneedles in a subject. The device includes a body, a microneedle coupled to the body, a reservoir coupled to the body, a first motor. The microneedle is positioned within a chamber at a distal end of the body. The reservoir includes a biomaterial fluid and is in fluid communication with the microneedle. The first motor is configured to activate the reservoir to expel the biomaterial fluid to the microneedle. The device also includes a temperature control assembly coupled to the reservoir and configured to set and maintain a temperature of the reservoir, a second motor coupled to the body and the microneedle, a microneedle size device coupled to the microneedle and configured to set a length of the microneedle extending from the chamber. Lastly, the device includes a user interface configured to receive input from a user to control the microneedle to penetrate the tissue to inject the biomaterial fluid into the tissue to generate an in-situ microneedle in the tissue.

### CROSS-REFERENCE TO RELATED APPLICATIONS

This application claims priority to U.S. Provisional Patent Application No. 63/397,690, filed Aug. 12, 2022, which is incorporated herein by reference in its entirety.

### TECHNICAL FIELD

This disclosure relates to methods and devices for providing in-situ formation of microneedles or structures and their use in medical applications.

### BACKGROUND

Microneedle arrays are a promising technology for transdermal drug delivery and body fluid sampling. Microneedles are often referred to as “microneedle patches,” “microneedle arrays” and by other similar terms. As one might expect, in a competitive marketplace, there are numerous embodiments and attendant nomenclature.

Generally, conventional microneedle arrays are provided as patches of micron-scaled medical devices. These are used to administer vaccines, drugs, and other therapeutic agents. While microneedles were initially explored for transdermal drug delivery applications, their use has been extended for the intraocular, vaginal, transungual, cardiac, vascular, gastrointestinal, and intracochlear delivery of drugs. Microneedles are constructed through various methods, usually involving photolithographic processes or micromolding. These methods involve etching microscopic structure into resin or silicon in order to cast microneedles. Conventional microneedles are made from a variety of material ranging from silicon, titanium, stainless steel, and polymers. Microneedles range in size, shape, and function but are all used as an alternative to other delivery methods like the conventional hypodermic needle or other injection apparatus.

Generally, a microneedle array is a collection of microneedles, ranging from only a few microneedles to several hundred, attached to an applicator, sometimes a patch or other solid stamping device. The arrays are applied to the skin of patients and are given time to allow for the effective administration of drugs. Microneedles are an easier method for physicians as they require less training to apply and because they are not as hazardous as other needles, making the administration of drugs to patients safer and less painful while also avoiding some of the drawbacks of using other forms of drug delivery, such as risk of infection, production of hazardous waste, or cost.

However, various challenges limit the translation of many prefabricated microneedle arrays to clinical applications including (i) limited penetration, (ii) the requirement of customization, (iii) difficulties in achieving clinically relevant dimensions, (iv) limited conformation to skin, (v) the need for secondary bandages to secure the array in place, (vi) inability of simultaneous administration of various therapeutics with controlled spatiotemporal distribution, and (vii) limited shelf-life of the microneedle arrays. Accordingly, materials, devices, and methods that can alleviate the aforementioned problems would be useful.

### SUMMARY

The present disclosure provides embodiments generally directed to a handheld device for forming in-situ microneedles in a tissue. In one embodiment, the present disclosure provides a device for forming in-situ microneedles in tissue. The device comprises a body, a microneedle coupled to the body, the microneedle positioned within a chamber at a distal end of the body, a reservoir coupled to the body, the reservoir including a biomaterial fluid, the reservoir in fluid communication with the microneedle a first motor coupled to the body and the reservoir, the first motor configured to activate the reservoir to expel the biomaterial fluid to the microneedle, a temperature control assembly coupled to the reservoir, the temperature control assembly configured to set and maintain a temperature of the reservoir, a second motor coupled to the body and the microneedle, a microneedle size device coupled to the microneedle and configured to set a length of the microneedle extending from the chamber; and a user interface configured to receive input from a user to control the microneedle to penetrate the tissue to inject the biomaterial fluid into the tissue to generate an in-situ microneedle in the tissue.

In another embodiment, the present disclosure provides a device for forming in-situ microneedles in tissue. The device comprises a body, a microneedle coupled to the body, a first motor coupled to the body, a second motor linked to the microneedle, the second motor configured to move the microneedle to puncture the tissue, and a controller coupled to the body, the controller in communication with the first motor and the second motor, the controller configured to coordinate activation of the first motor and the second motor to expel a biomaterial fluid from a reservoir to the microneedle and into the tissue to form an in-situ microneedle.

In yet another embodiment the present disclosure provides a device for forming in-situ microneedles in tissue. The device comprises a controller, an eccentric motor in communication with the controller, the eccentric motor linked to a microneedle, the controller configured to activate the eccentric motor to provide a reciprocating motion to the microneedle, and a reservoir in fluid communication with the microneedle, the reservoir including a biomaterial fluid, wherein the microneedle is configured to puncture the tissue to deliver the biomaterial fluid from the reservoir into the tissue and form an in-situ microneedle in the tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows an example concept of in-situ microneedle formation device for drug delivery.

FIG. 1B illustrates aspects of in-situ microneedle formation using biomaterial ink (also referred to as “biomaterial fluid” and “biomaterial precursor” herein) for controlled release of therapeutics for wound healing. Also shown is a dense microneedle array formed in-situ within a skin model with high aspect ratio needles.

FIG. 1C is a schematic of the device illustrated in FIG. 1A.

FIG. 2A is a front side view of an example of an in-situ microneedle formation device.

FIG. 2B is a rear side view of an example of an in-situ microneedle formation device.

FIG. 3 shows different views of an example housing door of an in-situ microneedle formation device.

FIG. 4 shows images of example needle embodiments. In FIG. 4 (at A), a tubing is coupled to a microneedle to enable delivery of the biomaterial precursor from the needle chamber. FIG. 4 (at B) depicts an array of hollow needles. FIG. 4 (at C) depicts an array of solid needles. In FIG. 4 (at D) a housing for the hollow needle array is depicted.

FIG. 5 shows an example of an in-situ microneedle formation device.

FIG. 6A shows the ability to control the formation of in-situ microneedle arrays with embodiments of the present disclosure. FIG. 6A (top row) depicts a side view of a series of microneedles formed in-situ at varying depths and in a range of biologically relevant sizes. FIG. 6A (middle row) depicts a side view of the in-situ formation of microneedles with different needle densities (frequencies). FIG. 6A (bottom row) depicts a top view of a series of microneedles formed in-situ with varying array sizes.

FIG. 6B graphically illustrates a quantitative measurement of the depth of the in-situ formed microneedles shown in the top row of FIG. 6A.

FIG. 6C graphically illustrates a quantitative measurement of the density of the in-situ formed microneedles shown in FIG. 6A (middle row).

FIG. 6D graphically illustrates a quantitative evaluation of a width of the needle arrays including 1, 5, 7, and 11 needles.

FIG. 7A shows various views of the formation of complicated microneedle geometries with high precision and consistency in penetration depth and microneedle frequency using an in-situ robotic microneedle delivery.

FIG. 7B depicts a top view of the formation of large and small microneedles structures with high accuracy in a single run using an in-situ robotic microneedle delivery.

FIG. 8A depicts representative images of different shapes formed through in-situ microneedle formation using an in-situ robotic microneedle delivery and the effect of needle configuration on the resolution and time of the treatment administered.

FIG. 8B graphically illustrates the resolution of a microneedle patch as a percentage of designed shape area shown in FIG. 8A.

FIG. 8C graphically illustrates the duration of the treatment administered with the microneedle patch designs shown in FIG. 8A.

FIG. 9A depicts representative images showing the formation of hybrid microneedle arrays in-situ. These images demonstrate the ability to fabricate microneedle arrays with various materials, length, and densities for dual delivery of drugs with different spatiotemporal profiles.

FIG. 9B graphically illustrates depth of microneedles shown in FIG. 9A (top graph) and distance between adjacent microneedles shown in FIG. 9A.

FIG. 9C (left side images) depict the formation of multi-angle microneedle arrays and the ability to control the spatial distribution and the adhesion of a microneedle patch to tissue. FIG. 9C (right side image) depicts an example of a multi-material microneedle patch with pre-designed architecture using an in-situ robotic microneedle delivery.

FIG. 10A graphically illustrates various rheological properties of biomaterial inks applicable for in-situ microneedle formation.

FIG. 10B graphically illustrates the delivery volume of biomaterial inks by changing the microneedle configuration.

FIG. 10C graphically illustrates release kinetics of biomaterial inks used for in-situ microneedle formation.

FIG. 11 depicts the delivery of complex particles and live cells through in-situ microneedle formation. FIG. 11 (at A) depicts the formation of microneedles encapsulating ZnO tetrapods inside of a model tissue. A zoomed-in image demonstrates the integrity of the particles delivered using the approach. FIG. 11 (at B) depicts live/dead staining (green/red, respectively) was used to evaluate the viability of the cells after implantation. The staining demonstrates the viability of more than 95% of the cells after implantation.

FIG. 12A (top row) depicts inhibition of microbial activity via in-situ provided microneedles. More particularly, the top row of images depicts the prevention of superficial growth of C. albicans biofilm when CPF was administered topically via drop or via microneedles. FIG. 12A (bottom row) illustrates the presence of a calcofluor fluorescence signal, which indicates that microneedle delivery reduces the overall biofilm thickness and biofilm growth at depths lower than 1 mm (red polygons).

FIG. 12B graphically illustrates that no significant differences were observed in fungal biofilm 3D growth between the drop and the microneedle delivery methods at t=0 h.

FIG. 12C graphically illustrates that biofilm 3D growth was reduced only when CPF was administered via microneedles at t=48 h.

FIG. 13 depicts an example application using in-situ microneedles for suture replacement/reinforcement in an esophagus anastomosis procedure of a pig model. FIG. 13 (at A) schematically depicts in-situ microneedle formation for sealing the cut tissues and controlled release of regenerative factors to accelerate tissue healing. FIG. 13 (at B) schematically depicts the setup of the experiment for measuring the burst pressure of the sealed tissues using in-situ microneedling process. FIG. 13 (at C) graphically illustrates the measured burst pressure of the cut esophagus tissue sealed with gelatin methacryloyl (GelMA), which was applied topically or through in-situ microneedle formation. Greater than 15 kPa burst pressure after sealing with in-situ microneedle formation demonstrates the benefits of using microneedling for suture replacement/reinforcement.

FIG. 14 depicts the feasibility and safety of in-situ microneedle formation in vivo. FIG. 14 (at A) schematically illustrates animal models with a full-thickness skin injury receiving different treatments. FIG. 14 (at B) depicts a comparison of the results of treating animal models where (1) no treatment was applied, (2) PBS was used as a control ink, and (3) 10% GelMA was used as a test ink followed by photocrosslinking. FIG. 14 (at C) graphically illustrates the in-situ microneedles formed in tissue did not negatively impact wound closure over 7 days.

FIG. 15A schematically illustrates an example of in-situ microneedle formation for the enhancement of topically administered drugs. An example of using an in-situ microneedling device to disrupt necrotic tissue and deliver the therapeutic agents deep inside it for accelerated debridement is illustrates.

FIG. 15B illustrates qualitative (left side image) and quantitative (right side image) results of drug distribution when administrated topically or using in-situ microneedle formation process. A deeper distribution can be seen when the drug is applied using microneedles.

### DETAILED DESCRIPTION

Disclosed herein are techniques for implanting microneedles, or microneedle-like structures, in a patient. The microneedles disclosed provide a patient with therapies by enabling delivery of at least one biologically active agent. Techniques disclosed include use of biomaterials to form the microneedles. A device that is adapted for implanting the microneedles is provided.

The disclosed technology provides a device that can overcome present challenges by enabling precise and effective delivery of therapeutics into the skin. The advantageous techniques for delivery are accomplished by the in-situ formation of microneedle array-like structures made of biomaterials. In general, the biomaterials used may encapsulate therapeutics. In some embodiments, therapeutics may be delivered subsequent to formation of the microneedles. Generally, the device enables injection of biomaterial fluid into tissue to generate the in-situ microneedles in the tissue.

The disclosed device is adjustable to a desired injection frequency and microneedle size/organization to generate custom arrays of microneedles with biomaterials that penetrate tissue. The in-situ formation of microneedles with the disclosed device overcomes the biomaterial-dependent penetration strength limitations of traditional microneedles. The disclosed device uses needles to penetrate tissue, form the shape of the microneedle, and deliver a biomaterial fluid that can be crosslinked to form a microneedle in-situ. For example, a surgeon can use the disclosed device and control the delivery of the biomaterial microneedle arrays in a direct write format. The device can operate in a direct write format to generate the in-situ microneedles directly into the tissue of a patient without prior fabrication steps. Alternatively, the device can be integrated with robotic systems to enhance the accuracy of implantation of the microneedles and eliminate human errors that may be associated with a hand-held device. In addition, the device can include an integrated light system (e.g., blue light) to induce in-situ crosslinking of photo-crosslinkable biomaterial precursors (e.g., inks/fluids). In addition to microneedles, the disclosed device can provide 3D printing and 3D bioprinting generated scaffolds that can penetrate tissue and/or coat the scaffolds.

The disclosed technology can eliminate the need for complicated tools for the fabrication of microneedles, enable immediate microneedle delivery, and mitigate changes in the chemical, morphological, or mechanical properties of prefabricated microneedle arrays during storage and transportation. The in-situ formed microneedle arrays can conform to irregular morphologies of wounds and tissues without the need for secondary bandages to secure the microneedle array.

For drug delivery applications, the in-situ formed microneedles can enable a slow release of therapeutics to minimize the need for multiple injections. The use of light during application of the device to generate the in-situ formed microneedles can lead to biological functions such as antibacterial activity, immune modulation, etc. The disclosed device can enable the delivery of different molecules, nanoparticles, microparticles, cells, etc., in a wide range of biomaterial carriers.

Another benefit of the disclosed technology is its facile integration with stimuli-responsive drug delivery approaches. Microneedle arrays with desired geometries can be formed in-situ that can then be triggered to release associated drug payloads using various thermal, irradiative, physical, or chemical stimuli, either externally or as a reaction to changes in the body. As disclosed herein, fabrication of a microneedle patch is independent of the mechanism of action of the microneedles and any stimuli induction system. The separation of these aspects provides for a simpler approach to treatment than with previous prefabricated arrays. Also, since the disclosed device does not require a large microneedle backing, the in-situ formed microneedle array can be in closer communication with the stimuli. The smaller or even lack of microneedle backing can further enhance tissue regeneration by not disrupting the mechanical properties of the native tissue. Additionally, by using less material, any material-based inhibition is reduced where it is not required.

The disclosed device and methods thereof can benefit both researchers in the field of biomedical engineering as well as clinicians. The disclosed in-situ microneedle arrays could be used for a wide variety of applications such as in the treatment of burns or chronic wounds, muscle injuries, melanoma, cancer immunotherapy, skin abnormalities, oral infections, internal organ infections, vaccine delivery, and body-fluid sampling. They can also be used for delivery of cells, such as stem cells.

### 1. Definitions

Before discussing the techniques disclosed herein in greater detail, some context is provided.

As used herein, the term “biologically active agent” generally refers to a substance that can act on a cell, virus, tissue, organ, organism, or the like, to create a change in the functioning of the cell, virus, tissue, organ, or organism. Examples of a biologically active agent include, but are not limited to, drugs, pharmaceuticals, anti-microbial agents, cells, proteins, enzymes, chemicals, and nucleic acids (e.g., mRNA, DNA, etc.). A biologically active agent is capable of treating and/or ameliorating a condition or disease, or one or more symptoms thereof, in a subject. Biologically active agents may also include prodrug forms of the agent. In general, prodrugs are medications that turn into an active form once they enter the body. Prodrugs help improve the effectiveness of the medication.

As used herein, the term “effective amount” or “therapeutically effective amount” generally refers to an amount sufficient to effect beneficial or desirable biological and/or clinical results.

As used herein, the term “subject” generally includes humans and mammals (e.g., mice, rats, pigs, cats, dogs, and horses). Examples of subjects may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

As used herein, the terms “transdermal” and “transdermally” generally refer to the delivery of a material, e.g., biomaterial fluid, across skin, such as skin of a subject. Transdermal also includes the delivery of a material to specific area(s) and/or layer(s) of the dermis, which can be referred to as intradermal, as well as to the delivery of a material to a specific tissue associated with the dermis, e.g., connective tissue, vasculature, glands, nerves, and the like.

As used herein, the term “intralesional” and “intralesionally” generally refers to the delivery of a material into a wound bed or injury site within a tissue.

As used herein, the term “treatment” or “treating” generally refers to protection of a subject from a disease, such as preventing, suppressing, repressing, ameliorating, or eliminating the disease. Preventing the disease involves administering a microneedle to a subject prior to onset of the disease. Suppressing the disease involves administering a microneedle to a subject after induction of the disease but before its clinical appearance. Repressing or ameliorating the disease involves administering a microneedle to a subject after clinical appearance of the disease.

As used herein, the term “microneedle like structure” and other similar terminology generally refers to structures that provide or exhibit many of the features and properties of a conventional microneedle, while also referring to structures that are fabricated according to the teachings provided herein.

As used herein, the term “microneedling” and other similar terminology generally refers to a therapeutic or other process that involves application of microneedle technology, such as application of a microneedle array or implanting microneedles provided as disclosed herein.

As provided herein, the “device” disclosed may be used for “direct write” of microneedles in a subject.

As used herein, the term “in-situ” generally refers to an activity performed at a point of therapy, such as implantation or fabrication of a microneedle array into the wound of a subject.

As used herein, the term “strategy” generally refers to techniques for fabricating and/or using microneedles according to the teachings herein.

### 2. Example Devices

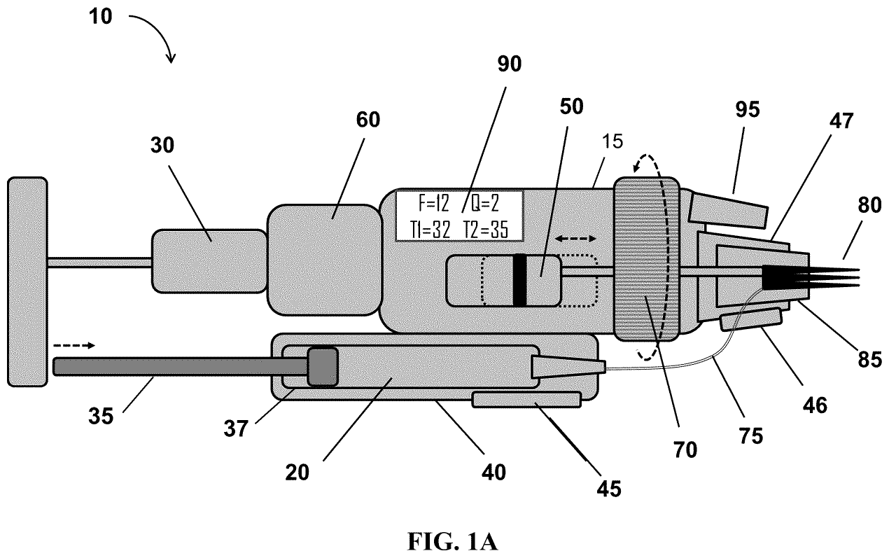

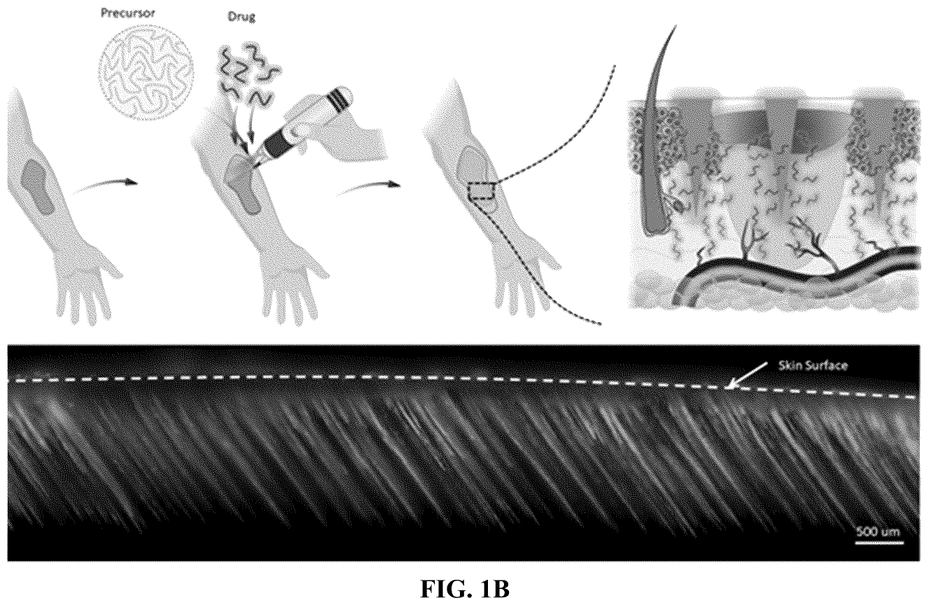

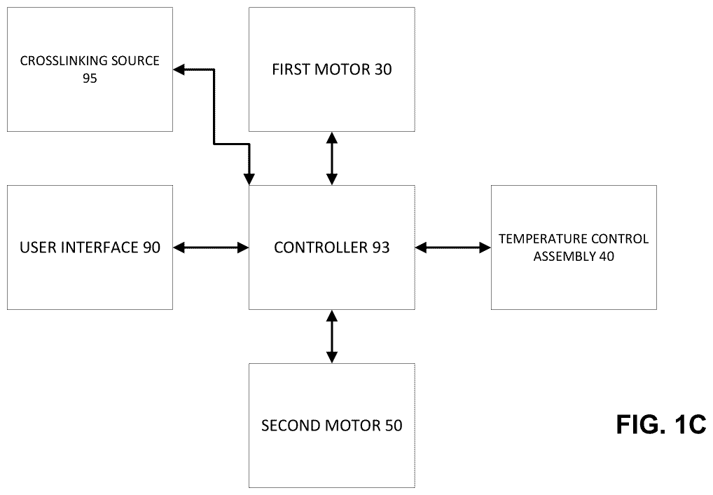

FIG. 1A schematically illustrates a device 10 for forming in-situ microneedles in tissue in accordance with an embodiment. In the illustrated embodiment, the device 10 is handheld and includes a body 15, a reservoir 20, also referred to as a “syringe,” a first motor 30, a temperature control assembly 40, a second motor 50, a power source 60, a microneedle size device 70, a microneedle 80, a user interface 90, and a controller 93. The device 10 can also include temperature sensors 45 and 46, a microneedle insulating case 47, a fluid conduit (e.g., tubing) 75, a microneedle chamber 85, and a crosslinking source 95.

The device 10 can be used to manually control the spatial deposition and formation of in-situ microneedle arrays in a target tissue by delivering a biomaterial fluid into micro-punctured tissue along with in-situ crosslinking. The biomaterial fluid can encapsulate drugs, vaccines, cells, bioactive factors, micro/nano particles, or combinations thereof (FIG. 1B). The device 10 can enable easy formation of microneedle arrays in desired density and penetration depth with a high aspect ratio. The delivery and application techniques described herein provides efficiently controlled spatiotemporal drug delivery and body-fluid sampling. FIG. 1B demonstrates a dense microneedle array with high aspect ratio formed in-situ within an in vitro skin model.

As shown in FIG. 1A, the reservoir 20 is coupled to the body 15 and can include a biomaterial fluid, also referred to as an “ink.” The biomaterial fluid can be used to encapsulate materials that can be released over time for desired therapeutic outcomes. A number of biomaterial fluids can be used in the disclosed devices and methods. For example, the biomaterial fluid can include a polymer. The polymer can be a synthetic polymer, a naturally occurring polymer (e.g., a protein) or a mixture thereof. The polymer can be functionalized with different chemicals and chemistries. The polymer can be capable of being crosslinked, e.g., after being deposited in tissue. The biomaterial fluid can have specific rheological properties such that once delivered into the tissue, it can form solid or gel-like structures.

The polymers, proteins, and their mixtures can be mixed with nanoparticles of any shape or microparticles of any shape or chemical and made from generally any material. Example materials include, but are not limited to, metal, metal oxides, bioglasses, radiopaque agents, antibacterial compounds and agents, antibiotics, bioceramics, ceramics, oxygen generating materials, proteins, vitamins, lipids, phospholipids, fatty acids, biological factors, polysaccharides, nucleic acids, growth factors, hydroxyapetite, carbon nanotubes, quaternary ammonium compounds, graphene, graphene oxide, carbon derived materials, liquid crystals, peptides, chitosan, silver nitride, platelet rich plasma, blood-derived materials and their combinations, etc. In some embodiments, the biomaterial fluid includes a biologically active agent, a particle-laden solution, or a combination thereof. In some embodiments, the biomaterial fluid includes a biologically active agent or a particle-laden solution. Some examples of biologically active agents include, but are not limited to, a nucleotide, a polynucleotide, a protein, a peptide, a carbohydrate, a lipid, a small molecule drug, chemicals, a cell, and combinations thereof.

The biomaterial fluid can have a varying viscosity. For example, the biomaterial fluid can have a viscosity of less than 1 Pa·s, less than 0.9 Pa·s, less than 0.8 Pa·s, less than 0.7 Pa·s, less than 0.6 Pa·s, or less than 0.5 Pa·s. The viscosity of the biomaterial fluid can be modulated by the environmental controller 30 (e.g., through temperature), the composition of the biomaterial fluid, or a combination thereof. In some embodiments, the biomaterial fluid has a shear thinning property.

The reservoir 20 can include one or more biomaterial fluids. For example, the reservoir 20 can include at least two, at least three, at least four, and so on different biomaterial fluids. In some embodiments, the reservoir 20 includes 1 to 6 different biomaterial fluids, such as 1 to 5 different biomaterial fluids, 1 to 3 different biomaterial fluids, 1 to 2 different biomaterial fluids, or 2 to 4 different biomaterial fluids. The reservoir 20 can include one or more separate compartments to accommodate the desired number of biomaterial fluids. For example, the reservoir 20 can include four separate compartments to accommodate four different biomaterial fluids.

The reservoir 20 and each compartment can have a varying volume. For example, the reservoir 20 can have a volume of about 0.1 mL to about 3 mL, such as about 0.5 mL to about 2.5 mL, about 1 mL to about 3 mL, or about 0.1 mL to about 2 mL. In some embodiments, the reservoir 20 has a volume of greater than 0.1 mL, greater than 0.5 mL, greater than 1 mL, or greater than 1.5 mL. In some embodiments, the reservoir 20 has a volume of less than 3 mL, less than 2.5 mL, less than 2 mL, or less than 1.5 mL. In some embodiments, each compartment in the reservoir 20 can have the same volume. In some embodiments, some compartments can have a different volume to accommodate different volumes of biomaterial fluids.

As illustrated in FIG. 1A, in an embodiment, the syringe 20 includes a plunger 35 and a barrel 37. The plunger 35 is coupled to the first motor 30, which is configured to activate the plunger 35 to move into the barrel 37. When the plunger 35 moves into the barrel 37, the biomaterial fluid is expelled from the barrel 37 through the fluid conduit 75 (e.g., Tygon® tubing) to the microneedle chamber 85 and/or the microneedle 80. The needle chamber 85 can hold the biomaterial fluid such that it can quickly and effectively be placed on a surface of the microneedle 80. The biomaterial fluid is then delivered in-situ by a reciprocating motion of the microneedle 80 relative to tissue.

The first motor 30 can cause the plunger 35 to move the biomaterial fluid into the microneedle chamber 85 and/or microneedle 80 at varying rates. For example, the first motor 30 can cause the plunger 35 to move the biomaterial fluid to the microneedle chamber 85 and/or microneedle 80 at about 0 mL/min to about 2 mL min, such as about 0.1 mL/min to about 2 mL/min, about 0.5 mL/min to about 1.5 mL/min, about 0.1 mL/min to about 1 mL/min, or about 1 mL/min to about 2 mL/min. In some embodiments, the first motor 30 can cause the plunger 35 to move the biomaterial fluid to the microneedle chamber 85 and/or microneedle 80 at greater than 0 mL/min, greater than 0.1 mL/min, greater than 0.5 mL/min, or greater than 1 mL/min. In some embodiments, the first motor 30 can cause the plunger 35 to move the biomaterial fluid to the microneedle chamber 85 and/or microneedle 80 at less than 2 mL/min, less than 1.5 mL/min, less than 1 mL/min, or less than 0.75 mL/min.

The temperature control assembly 40 includes a sleeve 43 and is coupled to the syringe 20 and may fully or partially cover the syringe 20. The temperature control assembly 40 is configured to control the temperature of the syringe 20 and the biomaterial fluid within the syringe 20. The temperature control assembly 40 can include at least one resistive element to control the temperature. Additionally, the temperature control assembly 40 may implement a cooling device, such as a loop of coolant provided by an external supply of coolant. The cooling device may be provided to accommodate temperature adjustments when, for example, changing from a first biomaterial fluid to a second biomaterial fluid.

For example, the temperature control assembly 40 can be configured to maintain the temperature of the syringe 20 at about 1° C. to about 150° C., such as about 10° C. to about 110° C., about 50° C. to about 100° C., about 1° C. to about 100° C., or about 75° C. to about 150° C. In some embodiments, the temperature control assembly 40 maintains the temperature of the syringe 20 at greater than 1° C., greater than 10° C., greater than 50° C., or greater than 100° C. In some embodiments, the temperature control assembly 40 maintains the temperature of the syringe 20 at less than 150° C., less than 125° C., less than 100° C., or less than 75° C. In some embodiments, the temperature control assembly 40 includes a thermal insulating case. To aid in accurate detection of the temperature, the temperature control assembly 40 can include a temperature sensor 45. In addition, the microneedle chamber 85 can be encased with a microneedle insulating case 47, which can include a temperature sensor 46. The microneedle insulating case 47 and the temperature sensor 46 can be in communication with the environmental control assembly 40 to maintain a desired temperature at the microneedle 80. In some embodiments, the microneedle insulating case 47 and the temperature sensor 46 mirror the temperature and/or range thereof of the environmental control assembly 40.

The temperature control assembly 40 can be used to maintain the biomaterial fluid at a specified temperature to aid in functioning of the device 10 and proper in-situ microneedle formation. For example, the temperature control assembly 40 can be configured to keep the biomaterial fluid at about 4° C. to about 80° C., such as about 10° C. to about 70° C., about 15° C. to about 60° C., about 20° C. to about 50° C., about 4° C. to about 50° C., or about 40° C. to about 80° C. In some embodiments, the temperature control assembly 40 maintains the biomaterial fluid at greater than 4° C., greater than 10° C., greater than 15° C., greater than 20° C., greater than 25° C., greater than 30° C., greater than 35° C., or greater than 40° C. In some embodiments, the temperature control assembly 40 maintains the biomaterial fluid at less than 80° C., less than 75° C., less than 70° C., less than 65° C., less than 60° C., less than 55° C., less than 50° C., or less than 45° C. To maintain the biomaterial fluid at a working temperature inside the syringe 20, which can be useful for thermally responsive biomaterials, plates (e.g., Peltier) or silicon heating pads can be incorporated.

The device 10 is configured to provide a back-and-forth movement (e.g., reciprocating motion) of the microneedle 80. The second motor 50 is supported by the body 15 and is coupled to the microneedle 80 with a link 55. The second motor 50 controls the back-and-forth movement of the microneedle 80 at a selected frequency and range of motion of the microneedle 80. In an embodiment, the second motor 50 can be an eccentric motor that can generate and control the frequency of the microneedle 80. The second motor 50 can include, but are not limited to, MOTOR-12V 10800RMP Mummy Customized Swiss Motor—With an Ultra Power No-Snag Motor and coreless Faulhaber motor rotary machine motor. The frequency of the microneedle 80 can be adjusted by tuning the supplied voltage to the second motor 50. The second motor 50 is configured to translate the rotational movement to linear movement. The working range of the microneedle 80 can be adjusted mechanically by using a screw. The screw adjusts the exposed length of the microneedle 80 for penetration into tissue.

The second motor 50 can control and generate movement of the microneedle 80 varying frequencies. For example, the second motor 50 can control and move the microneedle 80 at a frequency of about 2 Hz to about 200 Hz, such as about 10 Hz to about 175 Hz, about 50 Hz to about 150 Hz, about 2 Hz to about 100 Hz, or about 100 Hz to about 200 Hz. In some embodiments, the second motor 50 controls and moves the needle 80 at a frequency of greater than 2 Hz, greater than 25 Hz, greater than 50 Hz, or greater than 100 Hz. In some embodiments, the second motor 50 controls and moves the needle 80 at a frequency of less than 200 Hz, less than 150 Hz, less than 100 Hz, or less than 50 Hz.

The first motor 30 and the second motor 50 are coupled to the power source 60 to provide the energy needed to operate the device 10. The power source 60 also provides power for crosslinking, heating, and the like operations of the device 10. The power source 60 can be a battery or can plug into or connect to a mains power supply. In some embodiments, the power source 60 can supply power to the device 10 with one or more batteries (e.g., Nickel-Metal Hydride (NiMH), Nickel-Cadmium (NiCd), rechargeable lithium-ion, and the like) or an electrical plug cable for versatility of use. In some embodiments, batteries can provide an advantage of portability, which can make the device 10 convenient for use in a variety of settings. The batteries can include industry-standard features such as overcharge and short-circuit protection.

Alternatively, an electrical plug cable can allow continuous operation of the device 10 when access to a mains power supply is available. The power source 60, whether it be a battery or a plug cable, can be compatible with standard electrical systems across different regions, making the device 10 a practical tool for global application. In some embodiments, the power source 60 can be charged via wireless induction charging. The power source 60 can include a high-capacity battery. The battery can have a capacity of about 1800 mAh and can take a few hours (e.g., about 2 hours to about 3 hours) to fully charge, which can provide an operation time of over 5 hours, such as about 6 hours to about 8 hours. The power source 60 can also be charged via a USB C cable or other suitable cables.

The microneedle size device 70 is configured to adjust a size of the microneedle 80. For example, the microneedle size device 70 can include a knob to adjust the length of the microneedle 80. The knob is configured to rotate like a ratcheting mechanism to extend or retract the microneedle 80 from the microneedle chamber 85. The microneedle 80 progressively extends from the microneedle chamber 85 as the knob is turned to adjust the exposed length of the microneedle 80 for penetration into tissue. The microneedle size device 70 can be constructed from durable, wear-resistant materials, which can ensure sustained performance with repeated use. The knob can be ergonomically designed, which can promote ease of handling and precise control over the microneedle size. Additionally, the knob can allow for the facile modification of microneedle size to accommodate different user requirements and tissue types. These aspects can provide users of the device 10 with adjustability and options to accommodate various needs for and during medical treatment procedures.

The microneedle size device 70 can provide various exposed lengths of the microneedle 80 for piercing tissue. For example, the microneedle size device 70 can provide a length of the microneedle 80 of about 0.1 mm to about 4 mm, such as about 0.5 mm to about 3.5 mm, about 1 mm to about 3 mm, about 0.1 mm to about 2 mm, or about 2 mm to about 4 mm. In some embodiments, the microneedle size device 70 can provide a length of the microneedle 80 of greater than 0.1 mm, greater than 0.5 mm, greater than 1 mm, or greater than 2 mm. In some embodiments, the microneedle size device 70 can provide a length of the microneedle 80 of less than 4 mm, less than 3.5 mm, less than 3 mm, or less than 2.5 mm.

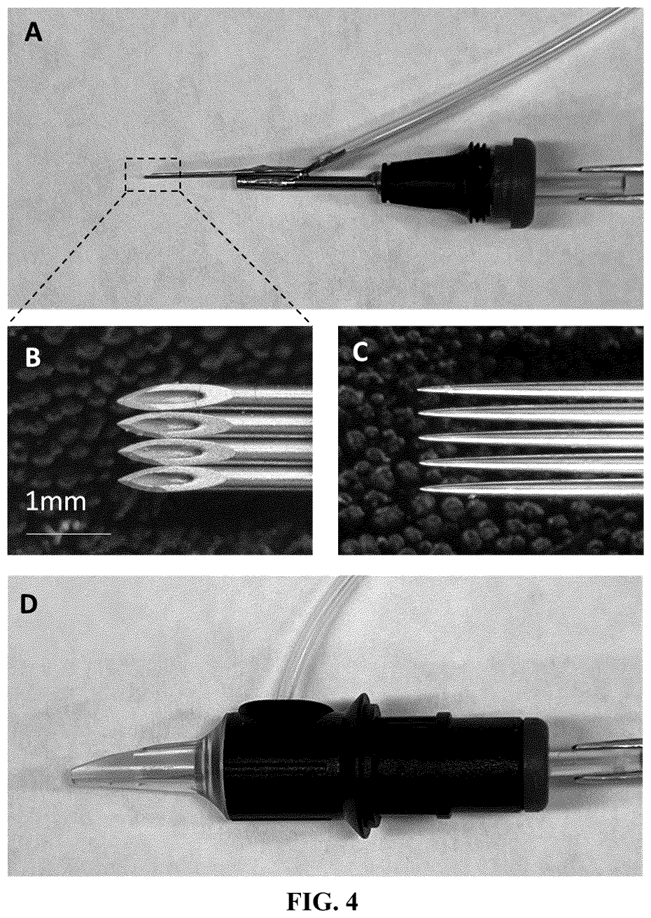

The microneedle 80 can be used to pierce an area of tissue in order to deposit the biomaterial fluid at the area and into the tissue. As shown in FIG. 1A, the biomaterial fluid can be transferred from the syringe 20 to the microneedle 80 through a fluid conduit 75, e.g., tubing, which can be controlled by the first motor 30. In other words, the microneedle 80 is in fluid communication with the syringe 20. The needle 80 can comprise one or more hollow (FIG. 4 (at B)) or solid needles (FIG. 4 (at C)).

In some embodiments, the microneedle 80 has a hollow core and can deliver the biomaterial fluid therethrough. Hollow needles provide effective delivery of the biomaterial fluid through the microneedle 80 without damaging the biomaterial fluid or its components (e.g., cells) by shear stress. FIG. 4 (at D) shows an example embodiment with an assembled hollow needle array and its housing. In addition, the microneedle 80 can be a single microneedle or a plurality of microneedles. For example, the microneedle 80 can include 1 microneedle, 2 microneedles, 5 microneedles, 11 microneedles, or more. In some embodiments, the microneedle 80 includes 1 microneedle to 20 microneedles.

The user interface 90 is supported on the body 15 and can include a display (e.g., a touch screen). The user interface 90 is accessed by a user to control the device 10. For example, the user interface 90 can be configured to control communication between the syringe 20, the first motor 30, and the microneedle 80. In some embodiments, the user interface 90 is configured to receive input from a user to control the microneedle 80. The user interface can receive input to control various parameters of the microneedle 80. For example, the user interface can be accessed to select a speed, a depth, a residence time, or a combination thereof of the microneedle 80.

As illustrated in FIG. 1C, the device 10 includes a controller 93 configured to process the input from the user interface 90 and to coordinate operation and functionality of the first motor 30, the second motor 50, and the microneedle 80. The controller 93 is also in communication with the temperature control assembly 40 to receive and monitor measured temperatures to maintain a temperature selected by the user on the user interface 90. The controller 93 is also in communication with the crosslinking source 95 to control activation of the source 95. The controller 93 can include an electronic processor and a memory. The memory may include, for example, a program storage area and a data storage area. The memory may include read-only memory and random-access memory.

With continued reference to FIG. 1A, the crosslinking source 95 is coupled to the body 15 and can provide a stimulus to crosslink the biomaterial fluid when used to provide in-situ microneedles. In an embodiment, the crosslinking source 95 can include a light, such as a blue light 405 nm LED, which can be powered by the power source 60. The device 10 is capable of creating microneedles from photocrosslinkable biomaterials, such as GelMA. The crosslinking source 60 is activated to photocrosslink the precursor biomaterials after injection into a tissue. Other example crosslinking sources 60 include, but are not limited to, ultraviolet light, infrared light, and laser light. Other crosslinking methods can include, but are not limited to, chemical, thermal, ionic, sound, enzymatic, and physical. The stimulation of the crosslinking can be supplied by a secondary source, e.g., incorporated into the original biomaterial deposited.

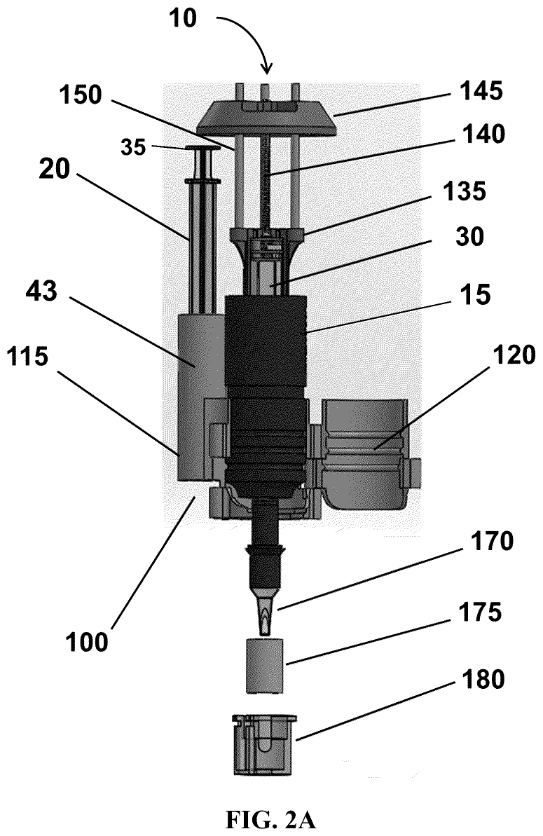

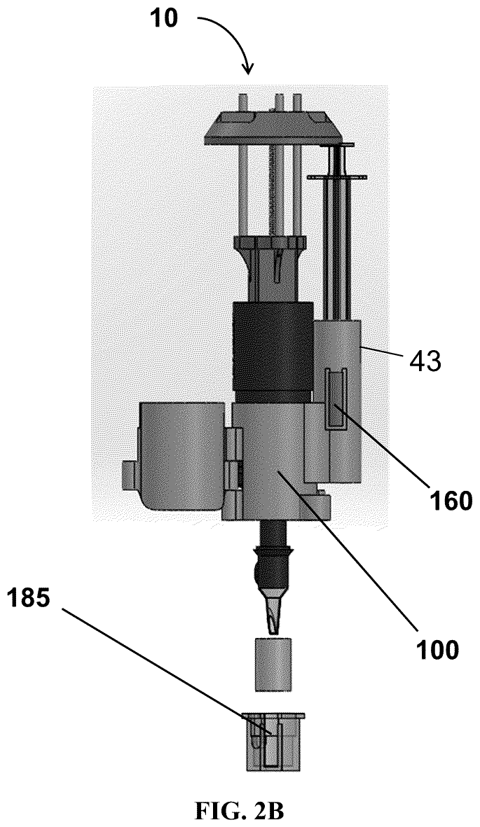

FIG. 2A and FIG. 2B illustrate the device 10 in more detail according to an embodiment. As illustrated, the device 10 is configured to be handheld. The device 10 includes an assembly 100 removably coupled to the body 15 of the device 10. The assembly 100 can include a door 120 having a hinge. The assembly 100 is configured to receive the body 15, and the door 120 can be closed to secure the body 15 within the assembly 100. The assembly 100 includes the temperature control assembly 40.

As illustrated in FIG. 2A, the temperature control assembly 40 includes the sleeve 43 configured to receive the syringe 20. The temperature control assembly 40 can include a cylindrical copper thermal jacket positioned inside the sleeve 43 that can interface with the syringe 20. The copper thermal jacket is conductive and can provide uniform heat transfer to and from the syringe 20 (and the biomaterial fluid within the syringe 20). The copper thermal jacket includes an outer surface where a resistive-based heater 110 can be coupled to the exterior surface to modify the temperature of the syringe 20 and the biomaterial fluid. In some embodiments, a Peltier thermoelectric jacket can be included in the sleeve 43 to expand temperature to sub-ambient temperatures. A desired temperature can be selected or set by accessing the user interface 90. In some embodiments, the temperature control assembly 40 can be positioned remote from the device 10.

With reference to FIG. 2B, the temperature control assembly 40 can include a digital logic (or electromechanical) circuit that can maintain the temperature at a specified setpoint as measured by a temperature sensor 160 on the sleeve 43 of the temperature control assembly 40. An example temperature sensor 160 includes, but is not limited to, a thermocouple. The setpoint can be correlated with a standard curve of measured temperatures within the syringe 20, which can account for discrepancies in ideal and measured temperatures. The temperature sensor 160 provides temperature measurements as feedback to the digital logic to maintain a temperature of the biomaterial fluid within ±0.05° C. of a specified temperature.

The syringe 20 can include or be filled with the biomaterial fluid. The biomaterial fluid can be delivered to the microneedle 80 by pushing the plunger 35 into the barrel 37 of the syringe 20. In some embodiments, the plunger 35 is removably coupled to a plate 145. The plate 145 is coupled to the first motor 30, which when activated causes the plate 145 to contact the plunger 35 to expel the biomaterial fluid from the syringe 20 to the microneedle 80. The first motor 30 can be positioned within a housing 135, which can be coupled to the body 15 of the device 10.

With continued reference to FIG. 2A, the first motor 30 generates rotational motion that is transferred to a shaft 140 (e.g., threaded shaft). The gear ratio between the first motor 30 and the shaft 140 can be varied to achieve different torque values. The rotation of the shaft 140 moves the plate 145 to contact the plunger 35 of the syringe 20. The plate 145 can include a threaded nut that rotates on the shaft 140 as the shaft 140 moves. The plate 145 can include one or more bushings (not shown) that are coupled to one or more linear guide rods 150, which are coupled to the housing 135. As the plate 145 moves along the shaft 140 and the linear guide rods 150, the plate 145 makes contact with the plunger 35 of the syringe 20 to expel the biomaterial fluid from the syringe 20 to the microneedle 80 via tubing or conduit 75.

With reference back to FIG. 2A, the device 10 can include a needle housing 170, a needle temperature control assembly 175, and a needle insulating case 180 to aid in temperature control of the microneedle 80 and the biomaterial fluid in the microneedle 80. The microneedle housing 170 can be configured to receive the microneedle 80. The insulating case 180 can be configured to receive the microneedle housing 170, the microneedle temperature control assembly 175, and the microneedle 80. The microneedle housing 170 is coupled with the microneedle temperature control assembly 175 to maintain the temperature of the biomaterial fluid during the micro-needling process. The microneedle temperature control assembly 175 can include a conductive thermal jacket. The temperature of the conductive thermal jacket can be controlled in the same or similar manner as the temperature control assembly 40. The microneedle temperature control assembly 175 can include a temperature sensor 185 coupled to an outer surface of the conductive thermal jacket to provide a closed-loop temperature control of the microneedle 80 and the biomaterial fluid. The insulating case 180 surrounds the microneedle temperature control assembly 175 to maintain the temperature of the microneedle 80 and the biomaterial fluid.

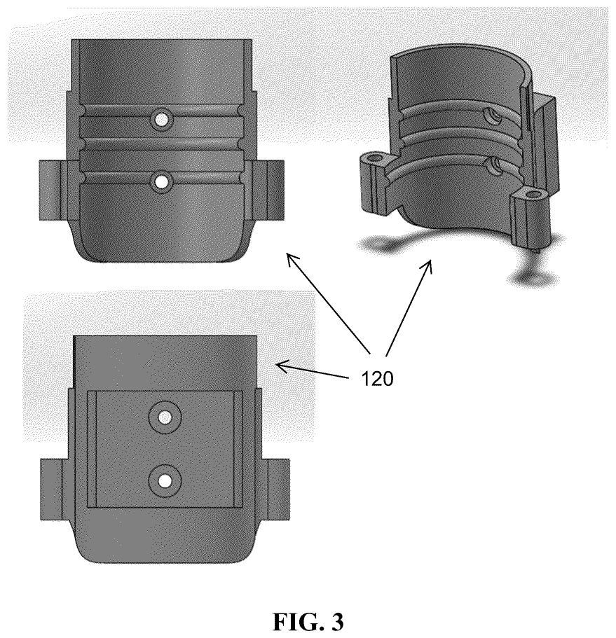

In alternative embodiments, the device 10 can be controlled by a user through automatic systems such as frame-based robots or robotic arms by mounting the device 10 on the robotic assembly. As shown in FIG. 3, the housing door 120 can be adapted to accommodate connection with the robotic assembly. For example, the housing door 120 can have two holes in the body of the housing door to engage with the robotic assembly. The robotic system is capable of utilizing computer-generated tooling paths (G-codes) from 3D or medical scans from a patient-specific area to guide movement and placement of the microneedle 80 to form the in-situ microneedles.

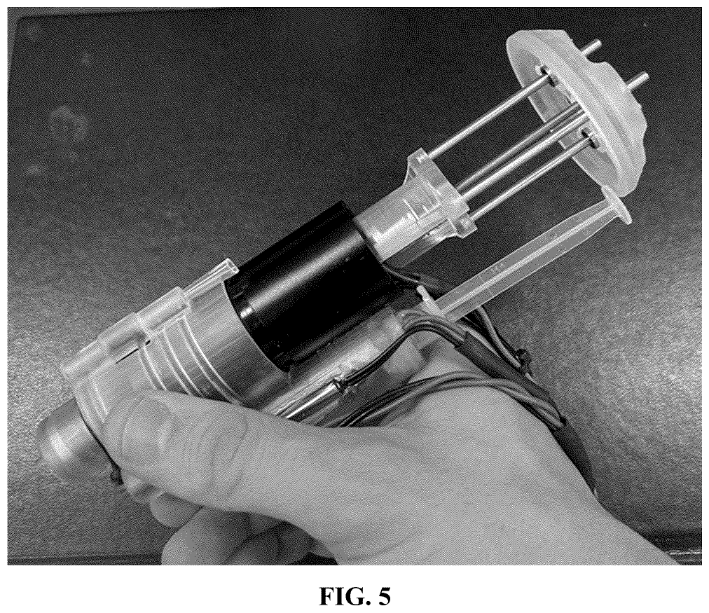

FIG. 5 depicts a prototype of the device 10 described above.

### 3. Example Methods

Also disclosed herein are methods for delivering a microneedle in-situ. Description of the example devices listed above can be applied to the methods of delivering microneedles in-situ. The method can include actuating (e.g., by an operator) the device 10 as disclosed herein to deliver the biomaterial fluid from the syringe 20 to the microneedle 80. The user can be an individual operating the device 10 as a handheld or the user may operate the device 10 through a robotic assembly.

The method can also include actuating a device 10 as disclosed herein to pierce an area of tissue with the microneedle 80. The user can actuate the device 10 by interacting with the user interface 90 to control the frequency of the microneedle 80. The user can adjust the microneedle size system 70 to set the length of the microneedle 80 for a desired depth of penetration of the microneedle 80 into tissue. The user can also input into the user interface 90 a residence time, i.e., a length of time the microneedle 80 remains in the tissue to deposit the biomaterial fluid. As can be seen, the user can actuate and control numerous parameters of the device 10 through the user interface 90.

Tissue to which the device is applied is not generally limited. In some embodiments, the tissue is the tissue of the subject or a tissue model. In some embodiments, the tissue is skin, vasculature, fibrosis, skeletal muscle, cardiac muscle, smooth muscle, subcutaneous (fat, collagen, nerves, etc.), tendon, cartilage, bone, eye, gum, dentin, stomach, esophagus, lung, stomach, colon, intestine, bladder, kidney, or liver.

Upon piercing the tissue (e.g., the surface of the tissue) with the microneedle 80, the biomaterial fluid can be deposited at the area (e.g., below the surface of the tissue). The method can further include crosslinking the biomaterial fluid to provide an in-situ formed microneedle at the area. The crosslinking can be mediated by physical crosslinking, thermal crosslinking, chemical crosslinking, ionic crosslinking, enzymatic crosslinking, a crosslinker, or a combination thereof. The crosslinking may be mediated by the crosslinking source 95. For example, in some embodiments, the device 10 includes a crosslinking source 95 that can be a source of blue light that can be used to crosslink the biomaterial fluid.

Because a single in-situ formed microneedle can be generated each time when the microneedle 80 pierces the tissue, the method can be repeated a number of times to provide a plurality of in-situ formed microneedles in the area. And, unlike traditional microneedle patches, the provided plurality of in-situ formed microneedles does not have to be interconnected through a backing material.

The microneedle 80 can have a varying depth (e.g., below the surface of the tissue). For example, the microneedle 80 can have a depth of about 1 μm to about 5 cm in the area, such as about 1 μm to about 2.5 cm, about 5 μm to about 2 cm, about 10 μm to about 1.5 cm, about 20 μm to about 1 cm, about 100 μm to about 500 mm, about 500 μm to about 100 mm, about 1 μm to about 1 cm, or about 500 μm to about 5 cm. In some embodiments, the microneedle 80 has a depth of greater than 1 μm, greater than 10 μm, greater than 100 μm, greater than 500 μm, greater than 1 mm, greater than 100 mm, greater than 500 mm, or greater than 1 cm. In some embodiments, the microneedle 80 has a depth of less than 5 cm, less than 1 cm, less than 500 cm, less than 100 cm, less than 1 mm, less than 500 μm, less than 100 μm, or less than 50 μm.

The microneedle can be formed at various angles in the tissue when the microneedle 80 is oriented at an angle relative to the surface of the tissue. For example, the microneedle can be formed at an angle relative to the surface of the tissue, of about 10° to about 90°, such as about 15° to about 80°, about 20° to about 75°, about 30° to about 60°, about 10° to about 60°, or about 30° to about 90°. In some embodiments, the microneedle 80 is formed at an angle of greater than 10°, greater than 20°, greater than 30°, greater than 40°, or greater than 50°. In some embodiments, the microneedle is formed at an angle of less than 90°, less than 80°, less than 70°, less than 60°, or less than 50° relative to the surface of the tissue.

The device 10 can deposit a varying amount of biomaterial fluid at the area. For example, the device 10 can deposit about 0.001 mL/in² to about 1 mL/in² of the biomaterial fluid at the area, such as about 0.01 mL/in² to about 1 mL/in², about 0.1 mL/in² to about 1 mL/in², about 0.001 mL/in² to about 0.5 mL/in², or about 0.001 mL/in² to about 0.1 mL/in² of the biomaterial fluid at the area. In some embodiments, the device 10 deposits greater than 0.001 mL/in², greater than 0.01 mL/in², or greater than 0.1 mL/in² of the biomaterial fluid at the area. In some embodiments, the device 10 deposits less than 1 mL/in², less than 0.5 mL/in², or less than 0.1 mL/in² of the biomaterial fluid at the area.

The in-situ formed microneedles at the area of a subject can be used to treat a disorder in the subject. The in-situ formed microneedles at the area of a subject can be used as a biosensor in the subject. The disorder is generally not limited and can be any disorder that may benefit from, e.g., therapeutics delivered by the microneedle, structure provided from the microneedle, or having biosensing provided from the microneedle. Example disorders include, but are not limited to, cancer, burns, diabetic ulcers, chronic wounds, oral wounds, oral infections, internal organ wounds, internal organ infections, muscle abnormalities, keloids, and combinations thereof. Example therapeutics include, but are not limited to, vaccines, contraceptive compounds, painkillers, anti-inflammatory compounds, immunomodulatory compounds, insulin, and combinations thereof.

The disclosed technology has multiple aspects, illustrated by the following non-limiting examples.

### 4. Examples

### Materials & Methods Used to Perform the Following Examples:

Effect of microneedling parameters on the organization of needles. Microneedles were generated on a 3% agarose skin model by encapsulation of fluorescent particles in bioink precursor (e.g., fluid/ink) and imaged on an inverted Zeiss Microscope at 5X objective. The device is equipped with a screw system that can continuously control the depth of microneedle penetration. Furthermore, the microneedle density can be controlled with the movement speed, and the microneedling frequency adjusted by controlling the input voltage. Finally, the width of the swipe path can be controlled by changing the integrated reservoir and needle unit. Post-processing of the images was done on ImageJ software.

Robotic arm integration. The handheld device was mounted on a robot arm (available from Rotrics) using a 3D printed adaptor. The adaptor was printed using an SLA printer (e.g., Form 3B, available from Formalabs). The ability of the device to form a microneedle patch with different structures utilizing three needle configurations (11F, 5F, and 1F) was tested. First, STL files of the geometries were designed in SolidWorks (available from Dassault Systemes). Then, each file was exported to an open-source slicing software (e.g., Ultimaker Cura; David Braam, Ultimaker), where printing parameters were fixed. Printing velocity was 10 mm/s and the size of each set of figures was determined based on the line width of each needle array. The line width for 11-, 5-, and 1-needle configurations was 3.75 mm, 1.6 mm, and 0.5 mm, respectively. To maintain the symmetry of the microneedle shapes, a height that was an odd multiple of each line width was selected. After establishing printing parameters, the code was sent to the robot arm and the time it took to form each structure was measured. The formed microneedle patches were investigated as described herein.

Rheological analysis. Rheological characterization of the various biomaterial inks was performed using a Discovery HR-3 hybrid rheometer (available from TA Instruments, USA). A 40 mm flat plate was mounted on the device head, the biomaterial inks were loaded, pre-shear was performed to remove any material memory from loading, and a continuous flow sweep with increasing shear rate was performed at 37° C. using an 800 μm gap. The viscosity by strain rate of each material was calculated by the Discovery HR-3 software.

Delivery Quantification. The capacity of the device to deliver different materials like hydrogels with a high degree of precision was evaluated by assessing the delivery of hydrogel containing rhodamine B (Rh-B). To perform the experiments, Rh-B (3,000 μg/mL) was mixed with a hydrogel solution (ink) at a 1:50 concentration, placed in a water bath at 39° C. for 4 min, and then loaded into the device. The ink used for this experiment was composed of 10% poly(ethylene glycol) diacrylate (PEGDA)+5% GelMA in PBS. The in-situ microneedling process was performed by carefully and continuously microneedling a straight line across a PDMS chamber filled with 20% gelatin for 15 sec. The amount of material delivered, and microneedles generated were tuned by changing the number of needles on each cartridge (5- vs 11-needle arrays) at a constant depth of 1 mm and a motor voltage of 5V. Once the microneedles were generated, the gelatin block was carefully removed from the PDMS mold, placed in a six-well plate, sealed to avoid evaporation, and placed in the dark until all the replications were generated. To melt the gelatin and homogeneously mix the delivered material, the well plate with the sample was placed in an oven for 10 min at 60° C. and subsequently placed on a hotplate at the same temperature with magnetic stirring. Once the pieces were homogenously mixed, 100 were quickly transferred into a 96-Well plate and read (excitation: 546 nm and a bandwidth of 9, emission: 568 nm and a bandwidth of 9) on a BioTek Cytation 5 plate reader (available from Agilent, USA) at 60° C. The samples for a standard curve were prepared similarly. The fluorescent signal was converted to the volume of gel delivered by referencing the original biomaterial ink concentration of Rh-B. The volume was normalized by the distance of the initial straight line using the initial PDMS chamber geometry.

Antifungal activity. The antifungal activity was performed using an in-lab developed 3D culture model. C. Albicans was seeded at a density of 10\hat{ }7 cells/mL and cultured for 48 h on CHROMagar. The 3D culture gels were treated with PBS and Caspofungin either topically or using the in-situ microneedle device and methodology. Samples were cut for in-depth imaging after 48 h of treatment and fungal inhibition was evaluated.

### Example 1

### Customizable In-situ Formation of Microneedles

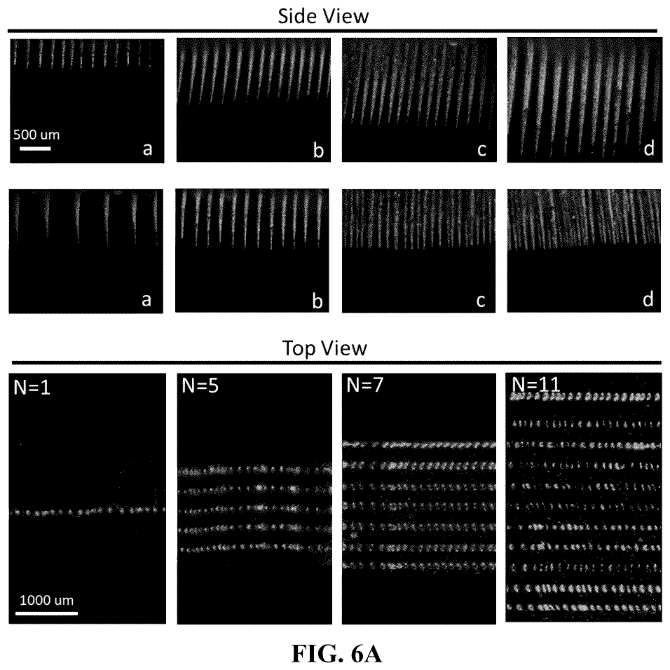

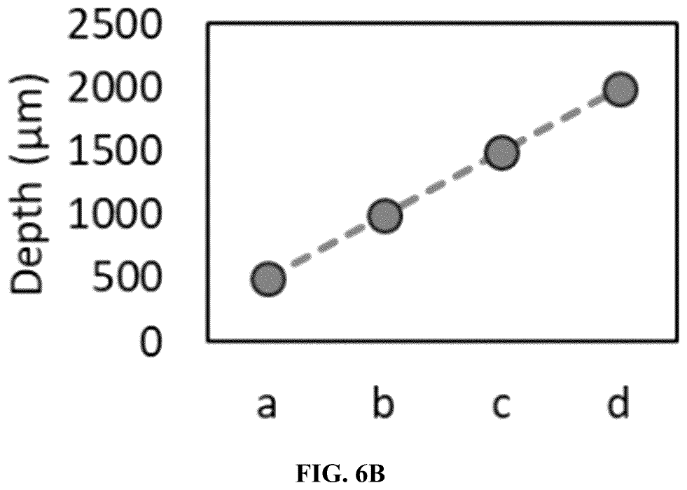

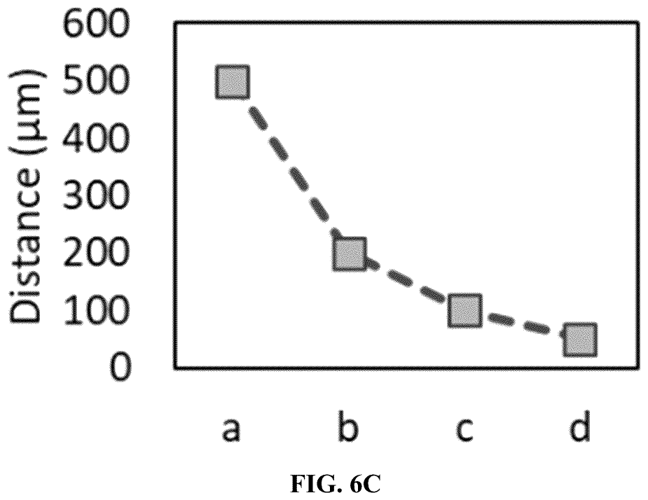

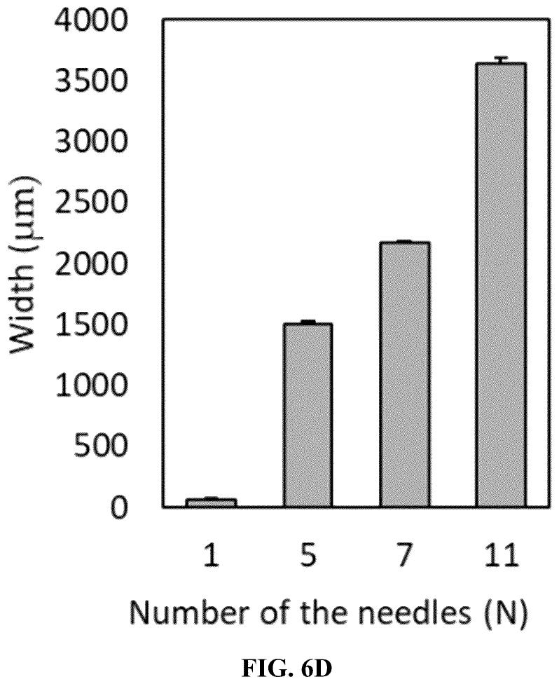

It is demonstrated that microneedles with controllable geometry can be formed in-situ inside the tissue with a broad selection of biomaterial fluids and inks. It is also demonstrated that microneedles fabricated out of very soft hydrogels can be implanted into a tissue. FIG. 6A (top row) and FIG. 6B illustrate facile and precise control of microneedle depth penetration, qualitatively and quantitatively. While the depth of microneedles can be controlled continuously from a few micrometers to multiple millimeters, a range of 500-2000 μm was considered here, which was relevant for intradermal delivery of therapeutics in, and sampling from human skin. While the device was set on a pre-designated needle length, a microneedle path with desired material and coverage shape was formed consistently. The density of the microneedles per area was easily modulated by controlling the microneedling frequency (FIG. 6A—middle row). Unlike conventional prefabricated microneedles which are limited to low-density needles, the results demonstrate that in the disclosed method, the needles can be highly dense, even contacting or overlapping each other, which results in higher interfacing area per administered patch. High control over the density of the microneedles enables high flexibility of the drug delivery strategy and adjustable tissue adhesion/integration and management.

Furthermore, needle organization were modulated to control the implantation speed and resolution over a specific area. As shown in FIG. 6C, a single needle organization was used to form linear arrays of microneedles, with high resolution and a width of less than a hundred micrometer (which is not trivial with prefabricated microneedles), while an 11-needle organization was implemented for generating around 3.5 millimeter wide strips of microneedle arrays for faster covering of the large areas (FIG. 6A—bottom row and FIG. 6D).

### Example 2

### Robotic Formation of Microneedles

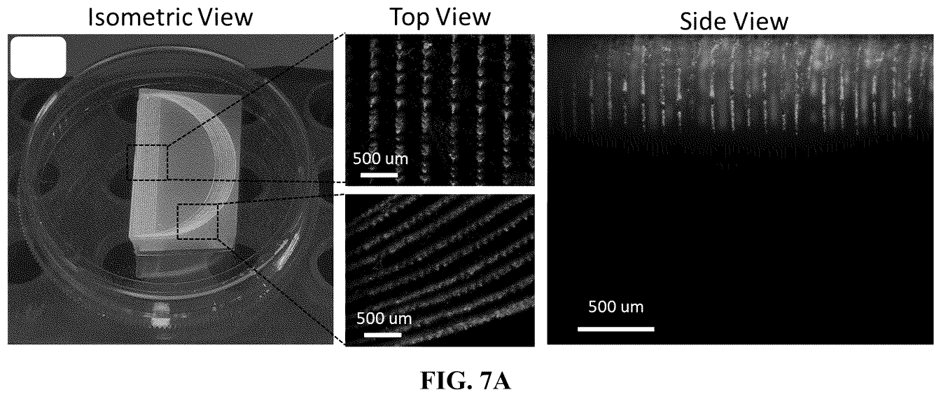

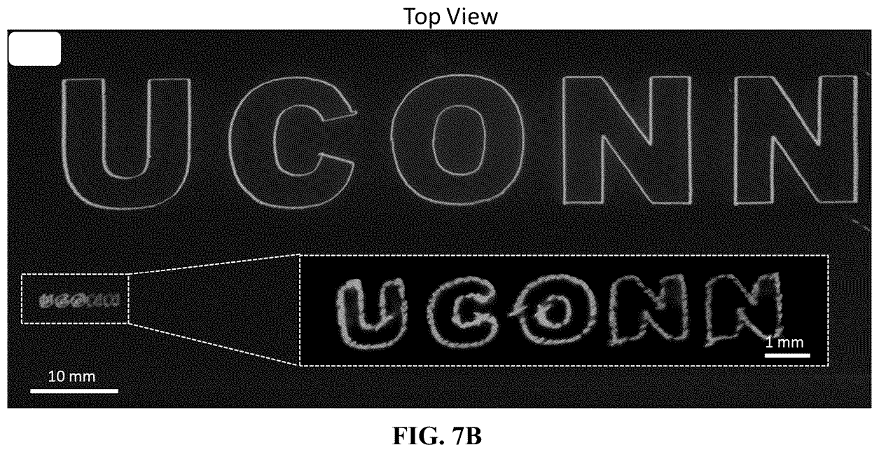

As mentioned elsewhere, the device can be mounted on an automated robotic assembly for enhancing the accuracy of implantation of the microneedles and eliminating human errors. A robotic arm was used to control the position of microneedle formation on an in vitro skin model. An 11-needle organization was used to form a microneedle array with a letter “D” structure, demonstrating the flexibility and accuracy of the strategy (FIG. 7A). The side view of the microneedling area demonstrates a high level of consistency in microneedle array density as well as size, as a result of robotic control. This strategy can be used to scan the defect area, reproduce the geometry in CAD software, and form desired microneedle area in-situ, recapitulating the defect structure. FIG. 7B illustrates the formation of patches having complex microneedle structures with high accuracy using the robotic arm. As shown, structures with different orders of magnitude in size can be easily formed e.g., simultaneously, using this system.

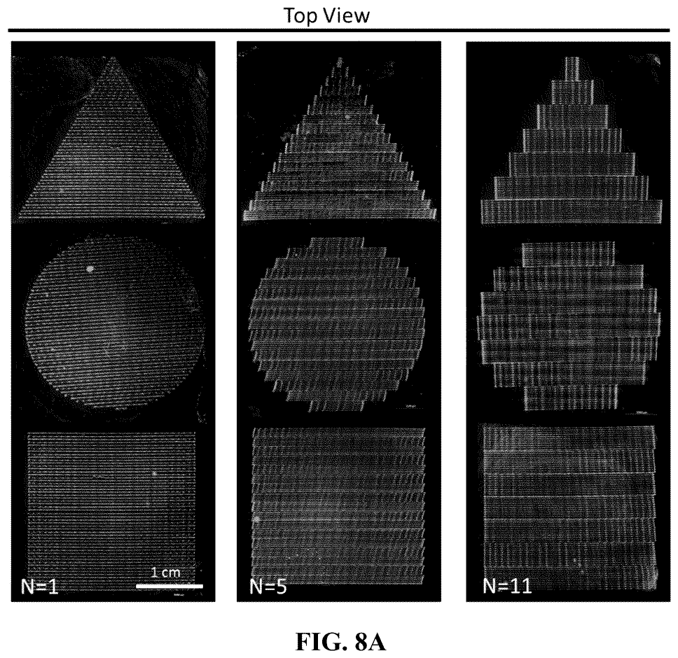

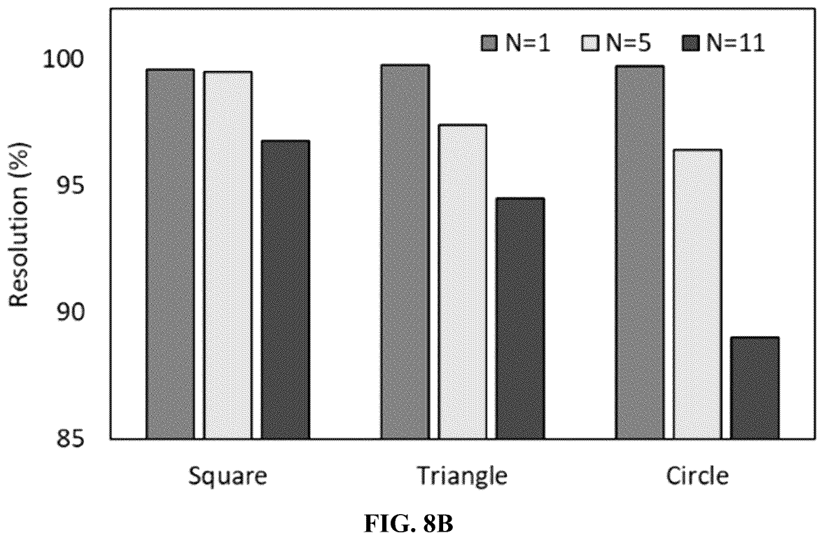

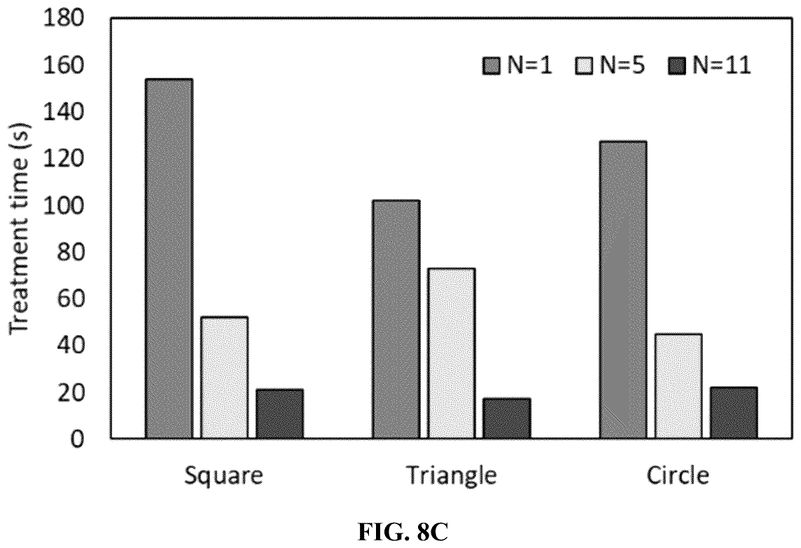

Using a robotic strategy, it was demonstrated that the microneedle organization can affect the resolution and duration of the treatment (FIG. 8A, FIG. 8B, and FIG. 8C). Three different needle configurations were used for this evaluation: 1-, 5-, and 11-needle arrays. As shown, the 1-needle configuration results in close to 100% resolution in all shapes, while the resolution decreases through the application of configurations with a higher number of needles. However, the resolution comes with a cost of a more time-consuming treatment. The treatment time in the experiments was around an order of magnitude less by replacing the 1-needle with the 11-needle configuration. This may be important when immediate care is required for large skin defects.

### Example 3

### Development of Hybrid Microneedle Patches

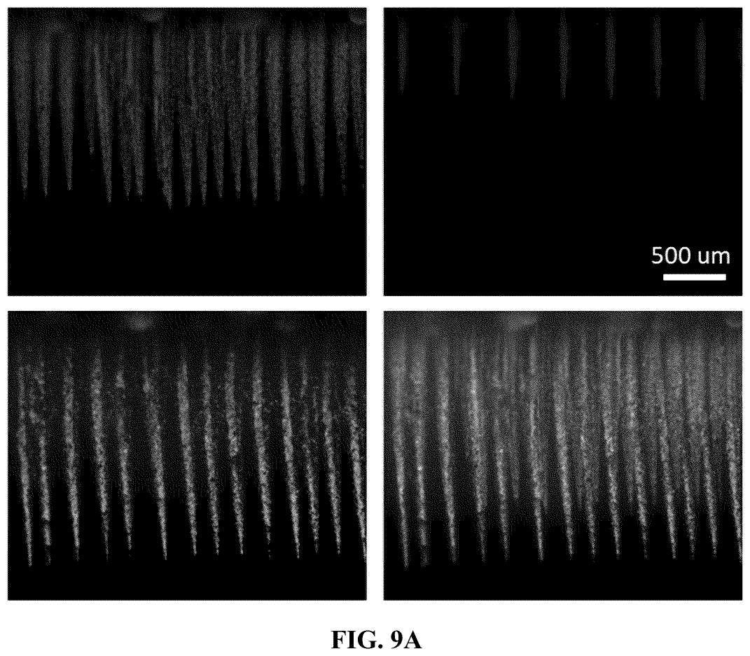

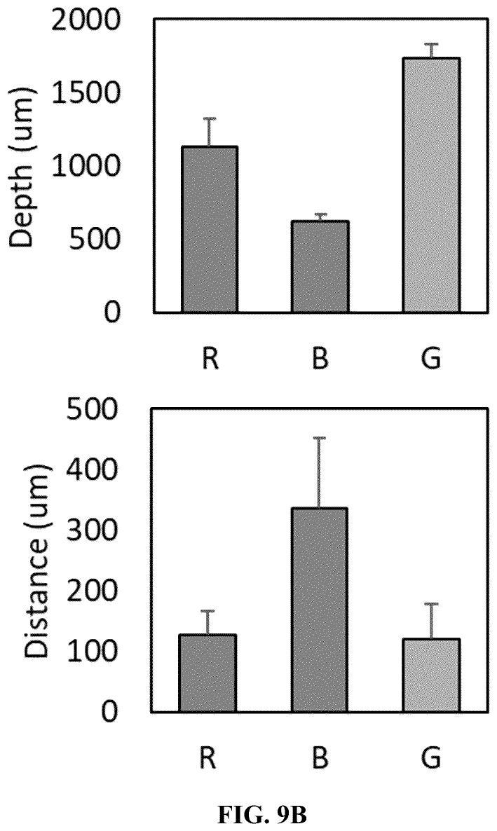

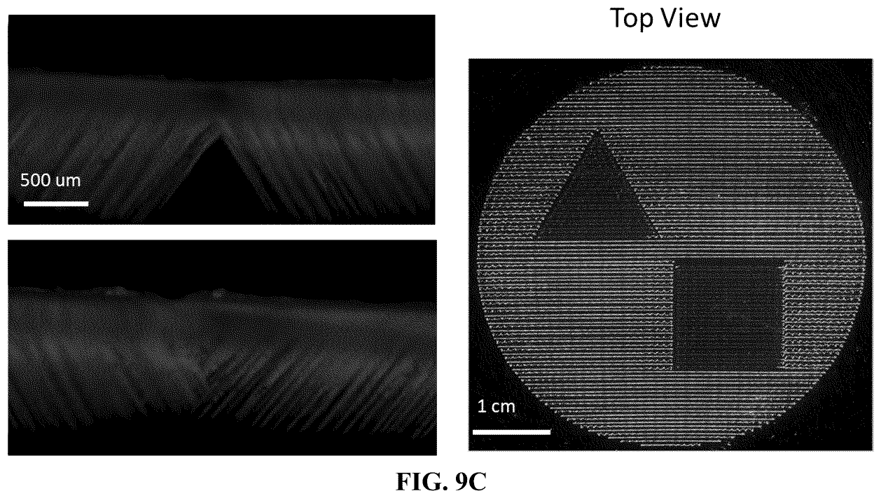

It is difficult to form microneedle arrays with high density, different microneedle sizes, and from different biomaterials, possibly encapsulating various drugs, has been difficult with previous methods. As described herein, multiple runs of in-situ formed microneedles provide the ability to implant arrays with high density, different microneedle sizes, and with different biomaterials, some with encapsulation of drugs. FIG. 9A-FIG. 9B demonstrate the formation of microneedle arrays with different biomaterial fluids or inks at different depths of a tissue model. The ability to generate these microneedle arrays is beneficial when the delivery of multiple therapeutics is required with a different spatiotemporal distribution. For example, in the treatment of infected chronic wounds, the smaller microneedles can fight infection by rapidly releasing antibacterial agents at more superficial levels while the longer needles can deliver angiogenic factors over longer periods deeper inside the wound bed to reach the vasculature and induce angiogenesis more effectively. In addition to the formation of multi-material and multi-length microneedle arrays, the disclosed technology enables the in-situ formation of microneedles with desired angles inside the tissue to both control the spatial delivery and microneedle array-tissue adhesion (FIG. 9C—left side image). Such structures with combined angled needles are very difficult to fabricate, and if fabricated, nearly impossible to penetrate inside the tissue without compromising the needle architecture and therefore its adhesion properties. This strategy can even be used for the adhesion of other implants to the tissues, specifically hydrogel implants which are nearly impossible to suture in place. Finally, hydrogels loaded with different drugs or cells can easily be fabricated in a customized manner over the defect area, creating precise planar control of drug delivery (FIG. 9C—right side image).

### Example 4

### Biomaterial Ink Characterization

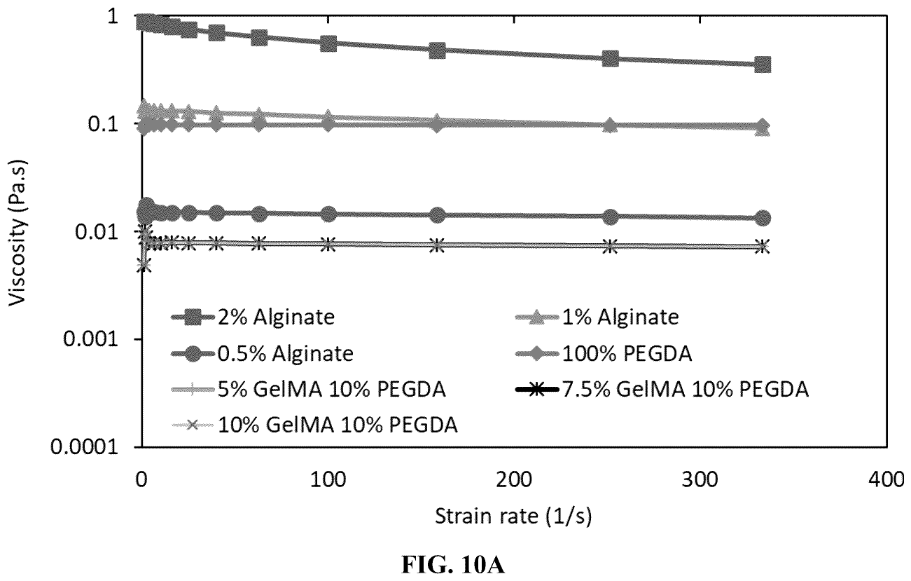

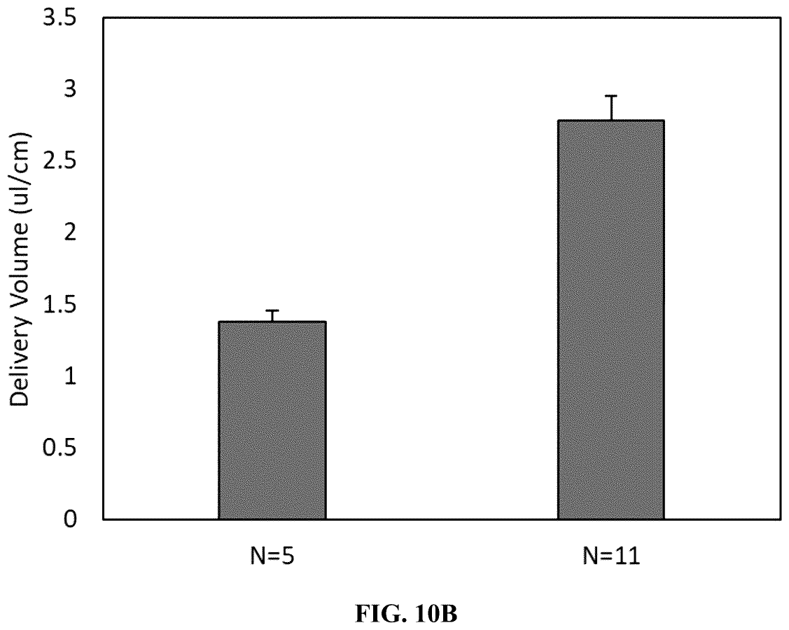

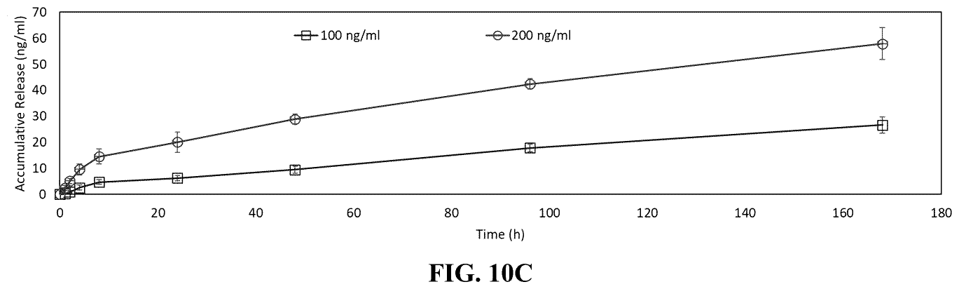

An advantage of the disclosed strategy is the easy implantation of the microneedle arrays. Unlike the prefabricated microneedle arrays, which have significant challenges associated with their penetration, specifically when using hydrogel biopolymers extensively used in tissue engineering and drug delivery applications, the disclosed strategy can deliver microneedles fabricated out of very soft hydrogels. FIG. 10A demonstrates the rheological properties of the inks that can be delivered using the disclosed strategy. As shown, inks with a viscosity of up to 1 Pa·s can be administrated using the method, though most polymer precursors do not have such high viscosities. Furthermore, the integration of the applicator with a blue light exposure system allows the crosslinking of microneedles made of photocrosslinkable materials such as GelMA, PEGDA, and/or N-isopropylacrylamide. The rheological properties of these hydrogels fall far below the limit of the administrable range (viscosity<0.1 Pa·s).

Here, a hybrid PEGDA/GelMA hydrogel was selected as an example biomaterial. This hybrid biomaterial is biocompatible, while the mechanical, physical, biological, and chemical properties of which are controllable. Specifically, through controlling the concentration of PEGDA and GelMA, biodegradability, cell permissibility, tissue adhesion/integration, and drug release kinetics can be controlled. This hybrid material, 5-10% GelMA, 5-10% PEGDA, was used.

The results demonstrate that the amount of delivered hydrogel can be precisely controlled by controlling the process parameters discussed above. FIG. 10B shows the control over the volume of hydrogel delivered with different needle configurations (5- vs 11-needle arrays). The control over the hydrogel volume provides the opportunity to control the drug delivery kinetics. Furthermore, it was demonstrated that the drug delivery kinetics can be adjusted using the concentration of the encapsulated drug. Here, vascular endothelial growth factor (VEGF), a biological agent that can enhance vascularization and therefore can aid in the treatment of acute and chronic wounds, was used. As shown, the microneedle biomaterial can provide a sustained long-term release of biomolecules for the treatment of skin defects (FIG. 10C).

### Example 5

### In-Situ Formation of Cell-/Particle Laden Microneedles

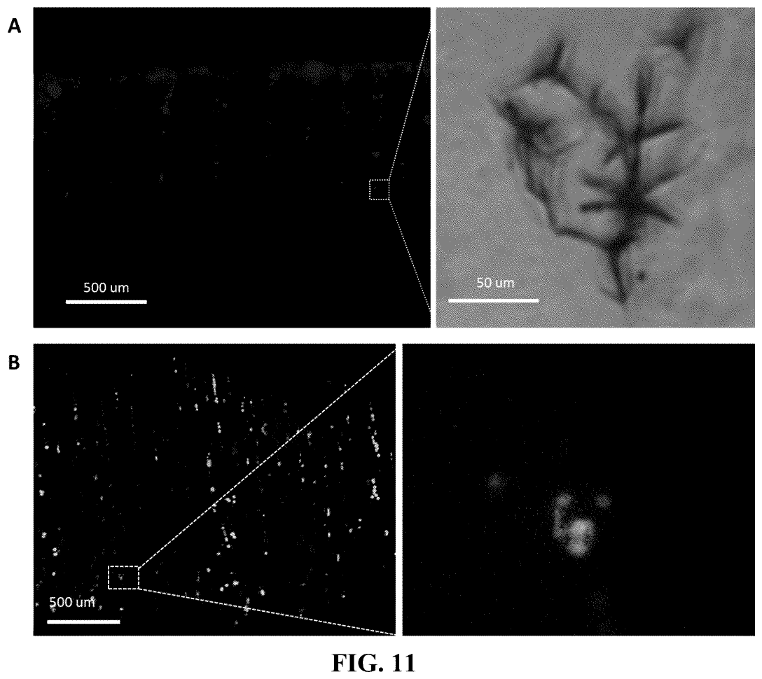

The capability of the disclosed strategy for delivering complex particles and live cells was demonstrated (FIG. 11). Here, hexapod ZnO particles, which have previously been shown to be beneficial for the treatment of skin infections, have been used (FIG. 11 (at A)). These particles could be delivered through encapsulation in precursor ink and in-situ formation of microneedles. Although these particles have a complex structure susceptible to fracture upon stress, which makes their application for microneedle-based therapy challenging, they easily withstood the applied stress during the administration with a disclosed microneedling device. Such particles can be used in the microneedle structure to enable antimicrobial activity, as well as to enhance tissue adhesion by anchoring to the surrounding tissue.

Live cell delivery was investigated using the disclosed approach. Cells were encapsulated in biomaterial ink and the microneedles were formed in-situ. (FIG. 11 (at B)) shows that most of the cells are alive (as indicated by green color vs. red dead cells) after in-situ microneedle formation. This is an important advantage of this technology since the delivery of live cells using previous microneedle arrays is very challenging due to the need for a biomaterial that is both cell-permissive and stiff enough to allow microneedle penetration into the tissue, two often opposing mechanical features. This simple strategy enables rapid and efficient delivery of cells deep inside the tissue. The application of hollow needle organization in the needle unit of the disclosed device further enhances the biocompatibility of the cell delivery in this approach by shielding the cells and other bioactive agents from high shear stresses that cause cell death.

### Example 6

### In-Depth Inhibition of Microbial Activity Through In-Situ Microneedle Formation

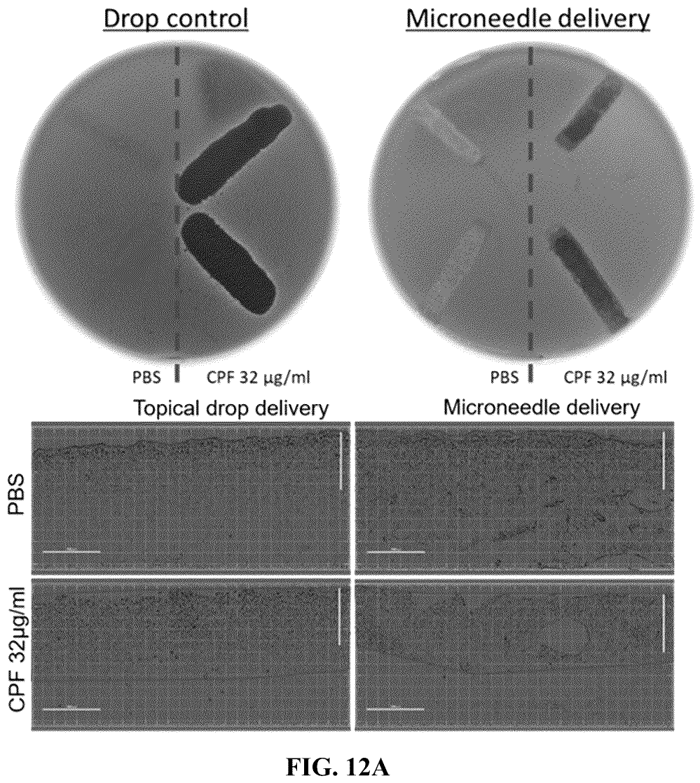

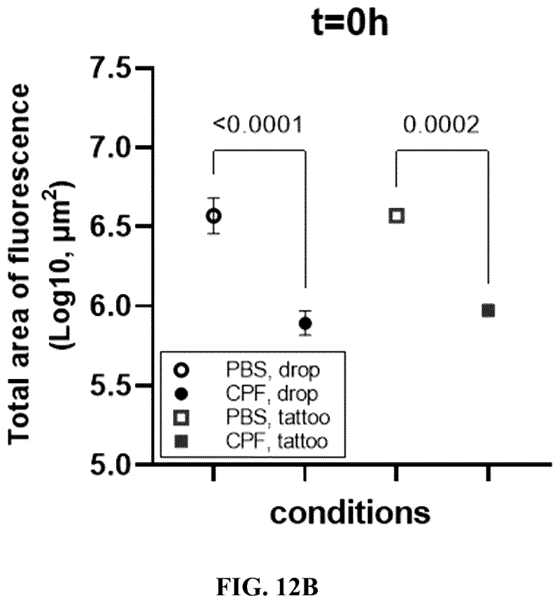

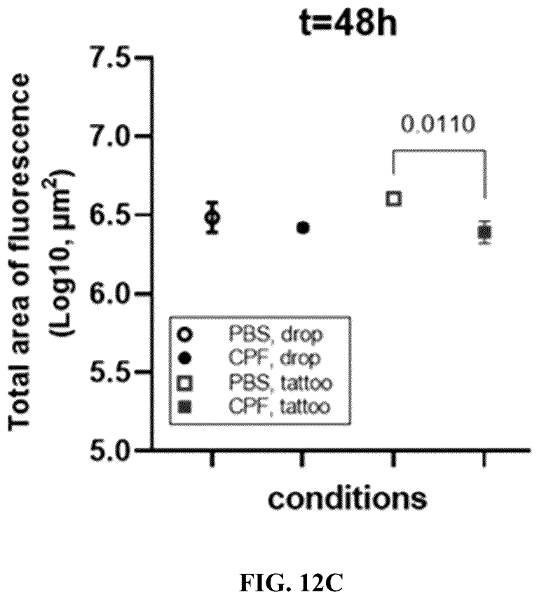

Experiments were performed to assess the feasibility of the disclosed device in the treatment of infection. 106 cells/ml of C. albicans were inoculated in liquid CHROMagar™ and allowed to form 3D fungal biofilms. PBS or 32 μg/ml of Caspofungin (CPF) was applied topically and via a microneedling device immediately after the agar solidified (t=0 h) or 48 h after biofilm growth (t=48 h). The growth and distribution of C. albicans was monitored for 48 hr. The preformed fungal biofilm (t=48 h) was not able to regrow on the surface after CPF was inoculated via drop and the microneedle system (FIG. 12A (top panel)). Fluorescence images of the slices show that CPF reduced the 3D growth when administered by both the drop and the microneedle system (FIG. 12A (bottom panel)). Interestingly, at t=48 h, CPF was effective in reducing the fungal biofilm growth only when administered via the microneedling system (FIG. 12B and FIG. 12C). These results suggest that microneedling may be a delivery method that can reduce systemic fungal dissemination from deep wounds, which is a significant source of morbidity and mortality.

### Example 7

### Application of In-Situ Microneedle Formation as a Fixation Modality

The beneficial application of in-situ microneedle formation as a suture replacement/reinforcement approach was investigated. As an example the strategy is used here for esophagus anastomosis.

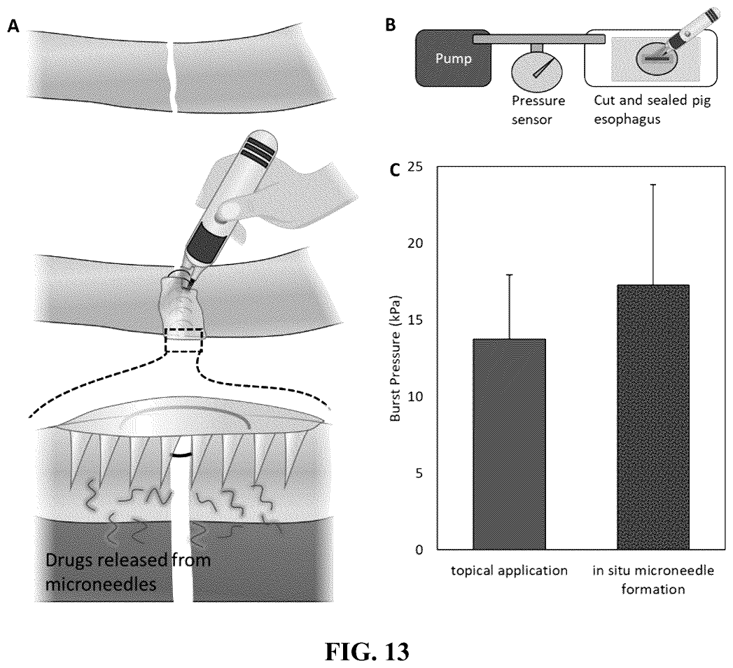

Esophageal cancer is the sixth leading cause of cancer mortality worldwide and is responsible for an estimated 508,600 deaths annually in 2018. Esophagectomy represents the gold standard approach for resection of locally advanced, diseased tissue and is followed by end-to-end anastomosis of proximal and distal esophageal segments to restore organ continuity. Despite advances in surgical techniques and perioperative care, overall postoperative morbidity remains high (56.0% at 30 days), with up to 26.9% of patients having a serious complication during treatment (Clavien Dindo classification grade Ma or higher). Postoperative anastomotic leaks (AL) represent one of the leading causes of death following esophagectomy with incidences as high as 35%. In addition, AL is associated with mediastinitis, sepsis, dysphagia, as well as strictures and often requires secondary surgical procedures and prolonged hospital stays to resolve anastomotic failures. The disclosed device was proposed to address the AL issue through in-situ formation of GelMA microneedles. FIG. 13 demonstrates the concept and the results on sealing cut porcine esophagus using the developed strategy. Preliminary results demonstrate the ability of the strategy to create a strong sealing and prevent AL. In this case, a scaffold like structure is generated that coats the tissue and parts of it penetrate the tissue.

### Example 8

### In Vivo Application of In-Situ Formed Microneedles

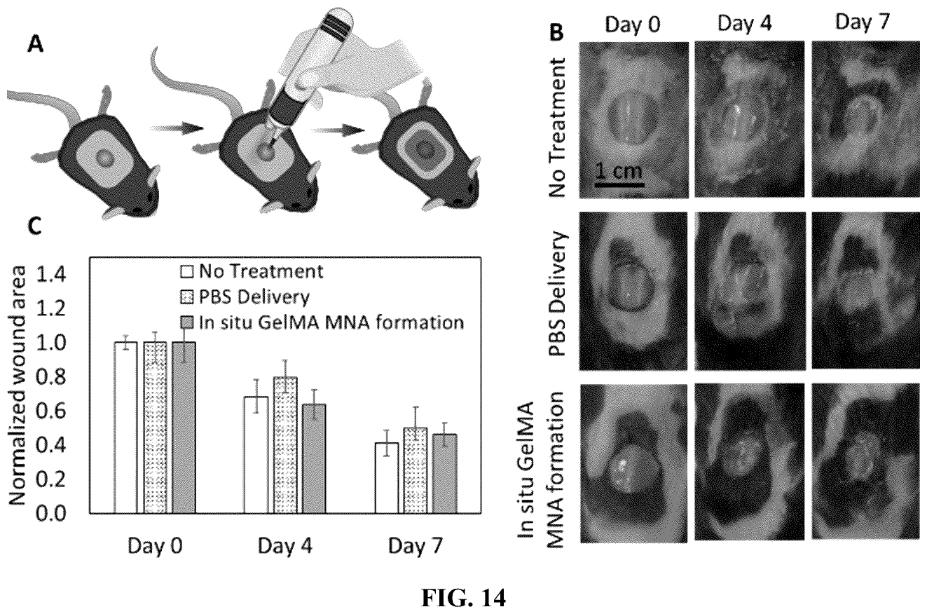

In another study, the potential negative effects of microneedling and in-situ formed microneedles were investigated in mice. 12-week-old C57BL/6 mice received a full-thickness skin cut on their dorsum. Animals were treated in three groups: 1) Tegaderm (negative control), 2) PBS delivery with in-situ microneedling (sham), and 3) in-situ formation of GelMA MNAs. Animals were then exposed to blue light photocrosslinking. The results demonstrated the safe application of in-situ formed MNAs with no negative impact on wound closure in gross analysis (FIG. 14).

### Example 9

### Application of In-Situ Microneedling to Enhance the Efficiency of Topically Administrated Drugs

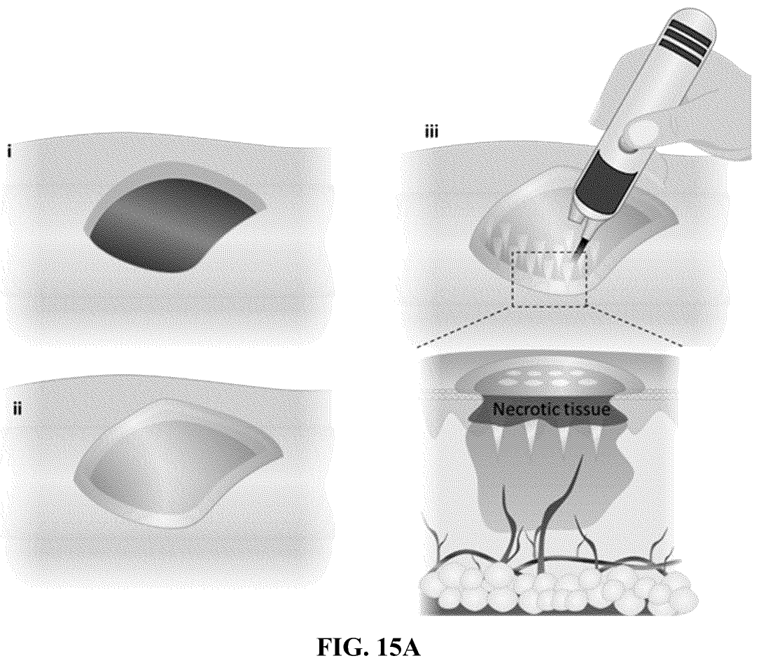

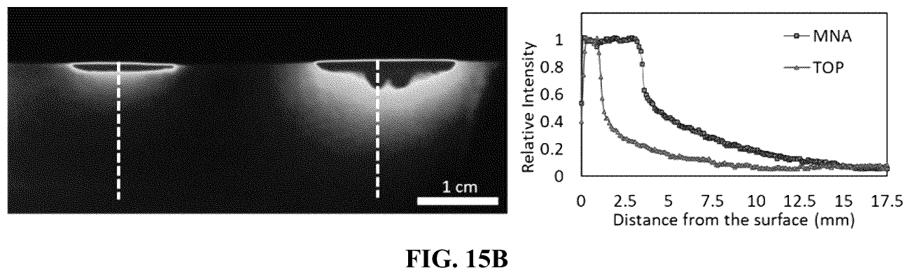

Microneedles can disrupt and bypass the tissue barriers, for example skin or eschar/necrotic tissue formed on top of the wounds. Therefore, the application of microneedling on an already administrated therapeutic can enhance the efficiency of the drug delivery. As an example, necrotic tissue debridement is currently being applied through highly invasive surgeries or less invasive topical applications. In the latter case, the wound is treated for multiple days with creams supplemented with collagenase, digesting the extracellular matrix, to wash out the necrotic tissue. However, the less invasive topical application usually has a limited effectiveness due to inefficient delivery of collagenase deep inside the necrotic tissue. Therefore, frequent application of the cream for a long time can be required. Here, we propose the application of in-situ microneedling on the topically applied therapeutic agent to disrupt the necrotic tissue and deliver the drug deep inside the necrotic tissue, accelerating the digestion of the necrotic tissue (FIG. 15A and FIG. 15B).

It is understood that the foregoing detailed description and accompanying examples are merely illustrative and are not to be taken as limitations upon the scope of the invention.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. The term “exemplary” is not intended to be construed as a superlative example but merely one of many possible examples.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

One or more embodiments are described and illustrated in the following description and accompanying drawings. These embodiments are not limited to the specific details provided herein and may be modified in various ways. Furthermore, other embodiments may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed.

For reasons of completeness, various aspects of the invention are set out in the following numbered clauses:

Clause 1. A device for forming in-situ microneedles in tissue, the device comprising: a body; a microneedle coupled to the body, the microneedle positioned within a chamber at a distal end of the body; a reservoir coupled to the body, the reservoir including a biomaterial fluid, the reservoir in fluid communication with the microneedle; a first motor coupled to the body and the reservoir, the first motor configured to activate the reservoir to expel the biomaterial fluid to the microneedle; a temperature control assembly coupled to the reservoir, the temperature control assembly configured to set and maintain a temperature of the reservoir; a second motor coupled to the body and the microneedle; a microneedle size device coupled to the microneedle and configured to set a length of the microneedle extending from the chamber; and a user interface configured to receive input from a user to control the microneedle to penetrate the tissue to inject the biomaterial fluid into the tissue to generate an in-situ microneedle in the tissue.

Clause 2. The device of clause 1, wherein the microneedle is configured to reciprocate and extend from a distal end of the device.

Clause 3. The device of clause 1 or 2, wherein the user interface is configured to receive input to control a speed, a depth, a residence time, or a combination thereof of the microneedle.

Clause 4. The device of any one of clauses 1-3, further comprising a controller configured to coordinate communication between the first motor, the second motor, and the microneedle.

Clause 5. The device of any one of clauses 1-4, further comprising an assembly removably coupled to the body, and wherein the reservoir and the temperature control assembly are coupled to the assembly.

Clause 6. The device of any one of clauses 1-5, wherein the reservoir includes a syringe, and wherein the temperature control assembly includes a syringe heater and a thermal insulating case, wherein the syringe includes the biomaterial fluid, and wherein the syringe heater is configured to provide heat to the syringe.

Clause 7. The device of any one of clauses 1-6, wherein the device is configured to be handheld by a user or the device is configured to be controlled by the user through a robotic assembly.

Clause 8. The device of any one of clauses 1-7, wherein the reservoir comprises at least two different biomaterial fluids.

Clause 9. The device of any one of clauses 1-8, wherein the temperature control assembly is configured to maintain a temperature of the biomaterial fluid at about 4° C. to about 80° C.

Clause 10. The device of any one of clauses 1-9, wherein the biomaterial fluid comprises a polymer.

Clause 11. The device of clause 10, wherein the polymer comprises a synthetic polymer, a naturally occurring polymer, or a combination thereof.

Clause 12. The device of any one of clauses 1-11, wherein the biomaterial fluid comprises a biologically active agent, a particle-laden solution, or a combination thereof.

Clause 13. The device of clause 12, wherein the biologically active agent comprises a nucleotide, a polynucleotide, a protein, a peptide, a carbohydrate, a lipid, a small molecule drug, a cell, or a combination thereof.

Clause 14. The device of any one of clauses 1-13, wherein the biomaterial fluid has a viscosity of less than 1 Pa·s or has a shear thinning property.

Clause 15. The device of any one of clauses 1-14, wherein the microneedle has a hollow core.

Clause 16. The device of any one of clauses 1-15, wherein the device includes a plurality of the microneedles.

Clause 17. The device of any one of clauses 1-16, further comprising a crosslinking source, and wherein the crosslinking source comprises a light source, an electrical current source, a heat source, a chemical source, an ion source, or a combination thereof.

Clause 18. A device for forming in-situ microneedles in tissue, the device comprising: a body; a microneedle coupled to the body; a first motor coupled to the body; a second motor linked to the microneedle, the second motor configured to move the microneedle to puncture the tissue; and a controller coupled to the body, the controller in communication with the first motor and the second motor, the controller configured to coordinate activation of the first motor and the second motor to expel a biomaterial fluid from a reservoir to the microneedle and into the tissue to form an in-situ microneedle.

Clause 19. A device for forming in-situ microneedles in tissue, the device comprising: a controller; an eccentric motor in communication with the controller, the eccentric motor linked to a microneedle, the controller configured to activate the eccentric motor to provide a reciprocating motion to the microneedle; and a reservoir in fluid communication with the microneedle, the reservoir including a biomaterial fluid; wherein the microneedle is configured to puncture the tissue to deliver the biomaterial fluid from the reservoir into the tissue and form an in-situ microneedle in the tissue.

Clause 20. The device of clause 19, further comprising a crosslinking source in communication with the controller, and wherein the crosslinking source is applied to the biomaterial fluid in the tissue to form the in-situ microneedle in the tissue.

Clause 21. A method of delivering a microneedle in-situ, the method comprising: actuating the device of any one of clauses 1-17 to place the biomaterial fluid on a surface of the needle; actuating the device to pierce an area of tissue with the needle, whereupon piercing the tissue with the needle, the biomaterial fluid is deposited at the area; and crosslinking the biomaterial fluid to provide a microneedle at the area.

Clause 22. The method of clause 21, wherein the microneedle has a depth of about 1 μm to about 5 cm in the area.

Clause 23. The method of clause 21 or 22, wherein the microneedle is formed at an angle of about 10° to about 90° relative to the tissue.

Clause 24. The method of any one of clauses 21-23, wherein the method is repeated to provide a plurality of microneedles in the area.

Clause 25. The method of clause 24, wherein the plurality of microneedles is not interconnected through a backing material.

Clause 26. The method of any one of clauses 21-25, wherein the crosslinking is mediated by physical crosslinking, thermal crosslinking, chemical crosslinking, ionic crosslinking, enzymatic crosslinking, a crosslinker, or a combination thereof.

Clause 27. The method of any one of clauses 21-26, wherein the device deposits about 0.001 mL/in² to about 1 mL/in² at the area.

Clause 28. The method of any one of clauses 21-27, wherein the device is controlled through a robotic assembly.

Clause 29. The method of any one of clauses 21-28, wherein the tissue includes a subject's tissue or a tissue model.

Clause 30. The method of clause 29, wherein the microneedle is used to treat a disorder in a subject, as a biosensor, or a combination thereof.

Clause 31. The method of any one of clauses 21-30, wherein the disorder comprises cancer, burns, diabetic ulcers, chronic wounds, oral wounds, oral infections, internal organ wounds, internal organ infections, muscle abnormalities, keloids, or a combination thereof.

Clause 32. The method of any one of clauses 21-31, wherein the microneedle delivers a vaccine, contraceptive compounds, painkillers, anti-inflammatory compounds, immunomodulatory compounds, or insulin.

Clause 33. A method of debriding tissue in a subject in need thereof, the method comprising actuating the device of any one of clauses 1-17 actuating the device to pierce an area of tissue with the needle, whereupon piercing the tissue with the needle removes part of the tissue and simultaneously delivers biomaterial fluid to the tissue.

Clause 34. The method of clause 33, wherein the biomaterial fluid comprises a polymer selected from the group consisting of a synthetic polymer, a naturally occurring polymer, and a combination thereof.

Clause 35. The method of clause 33 or 34, wherein the biomaterial fluid comprises a biologically active agent selected from the group consisting of a nucleotide, a polynucleotide, a protein, a peptide, a carbohydrate, a lipid, a small molecule drug, chemicals, nanoparticles, a cell, and a combination thereof.

Clause 36. A method of delivering a scaffold in-situ, the method comprising: actuating the device of any one of clauses 1-17 to place the biomaterial fluid on a surface of the needle; actuating the device to pierce an area of tissue with the needle, whereupon piercing the tissue with the needle, the biomaterial fluid is deposited at the area; and crosslinking the biomaterial fluid to provide a scaffold at the area.

Clause 37. The method of clause 36, wherein the scaffold is a fixation or reinforcement scaffold covering the tissue.

Clause 38. The method of clause 36 or 37, wherein the biomaterial fluid comprises a polymer selected from the group consisting of a synthetic polymer, a naturally occurring polymer, and a combination thereof.

Clause 39. The method of any one of clauses 36-38, wherein the biomaterial fluid comprises a biologically active agent selected from the group consisting of a nucleotide, a polynucleotide, a protein, a peptide, a carbohydrate, a lipid, a small molecule drug, chemicals, nanoparticles, a cell, and a combination thereof.

What is claimed is:

1. A device for forming in-situ microneedles in tissue, the device comprising:
a body;
a microneedle coupled to the body, the microneedle positioned within a chamber at a distal end of the body;
a reservoir coupled to the body, the reservoir adapted for a biomaterial fluid, the reservoir in fluid communication with the microneedle;
a first motor coupled to the body and the reservoir, the first motor configured to activate the reservoir to expel the biomaterial fluid to the microneedle;
a temperature control assembly coupled to the reservoir, the temperature control assembly configured to set and maintain a temperature of the reservoir;
a second motor coupled to the body and the microneedle;
a microneedle size device coupled to the microneedle and configured to set a length of the microneedle extending from the chamber; and
a user interface configured to receive input from a user to control the microneedle to penetrate the tissue to inject the biomaterial fluid into the tissue to generate an in-situ microneedle in the tissue.

2. The device of claim 1, wherein the microneedle is configured to reciprocate and extend from a distal end of the device to penetrate the tissue.

3. The device of claim 1, wherein the user interface is configured to receive input to control a speed, a depth, a residence time, or a combination thereof of the microneedle.

4. The device of claim 1, further comprising a controller configured to coordinate communication between the first motor, the second motor, and the microneedle.

5. The device of claim 1, further comprising an assembly removably coupled to the body, and wherein the reservoir and the temperature control assembly are coupled to the assembly.

6. The device of claim 5, wherein the reservoir includes a syringe, and wherein the temperature control assembly includes a syringe heater and a thermal insulating case, wherein the syringe includes the biomaterial fluid, and wherein the syringe heater is configured to provide heat to the syringe.

7. The device of claim 1, wherein the device is configured to be handheld by a user or the device is configured to be controlled by the user through a robotic assembly.

8. The device of claim 1, wherein the reservoir comprises at least two different biomaterial fluids.

9. The device of claim 1, wherein the temperature control assembly is configured to maintain a temperature of the biomaterial fluid at about 4° C. to about 80° C.

10. The device of claim 1, wherein the biomaterial fluid comprises a polymer.

11. The device of claim 10, wherein the polymer comprises a synthetic polymer, a naturally occurring polymer, or a combination thereof.

12. The device of claim 1, wherein the biomaterial fluid comprises a biologically active agent, a particle-laden solution, or a combination thereof.

13. The device of claim 12, wherein the biologically active agent comprises a nucleotide, a polynucleotide, a protein, a peptide, a carbohydrate, a lipid, a small molecule drug, a cell, or a combination thereof.

14. The device of claim 1, wherein the biomaterial fluid has a viscosity of less than 1 Pa·s or has a shear thinning property.

15. The device of claim 1, wherein the microneedle has a hollow core.

16. The device of claim 1, wherein the device includes a plurality of the microneedles.

17. The device of claim 1, further comprising a crosslinking source, wherein the crosslinking source comprises a light source, an electrical current source, a heat source, a chemical source, an ion source, a sound source, an enzymatic source, or a combination thereof.

18. A device for forming in-situ microneedles in tissue, the device comprising:
a body;
a microneedle coupled to the body;
a first motor coupled to the body;
a second motor linked to the microneedle, the second motor configured to move the microneedle to puncture the tissue; and
a controller coupled to the body, the controller in communication with the first motor and the second motor, the controller configured to coordinate activation of the first motor and the second motor to expel a biomaterial fluid from a reservoir to the microneedle and into the tissue to form an in-situ microneedle.

19. A device for forming in-situ microneedles in tissue, the device comprising:
a controller;
an eccentric motor in communication with the controller, the eccentric motor linked to a microneedle, the controller configured to activate the eccentric motor to provide a reciprocating motion to the microneedle; and
a reservoir in fluid communication with the microneedle, the reservoir including a biomaterial fluid;
wherein the microneedle is configured to puncture the tissue to deliver the biomaterial fluid from the reservoir into the tissue and form an in-situ microneedle in the tissue.

20. The device of claim 19, further comprising a crosslinking source in communication with the controller, and wherein the crosslinking source is applied to the biomaterial fluid in the tissue to form the in-situ microneedle in the tissue.
